(19) United States
(12) Patent Application Publication
(10) Pub. No.: US 2005/0154368 A1
(43) Pub. Date: Jul. 14, 2005

(54) Medical material handling systems

(75) Inventors: Bernard C.B. Lim, Oakville (CA); Taras Worona, Etobicoke (CA); Davis A.R. Kanbergs, Milton (CA); Mark Paul Costa, Milton (CA); Roy T. Zhao, Brampton (CA); Gabriele Klein, Mississauga (CA); Kathleen Muller, Mississauga (CA); David G. Matsuura, Encinitas, CA (US); Philip J. Simpson, Escondido, CA (US); Walter Dean Gillespie, San Diego, CA (US); John Norris Mitchell, Hertfordshire (GB); Hao Chen, Mississauga (CA)

Correspondence Address: BURNS DOANE SWECKER & MATHIS L L P, POST OFFICE BOX 1404, ALEXANDRIA, VA, 22313-1404, US

(73) Assignee: VASOGEN IRELAND LIMITED, County Clare (IE)

(21) Appl. No.: 10/991,447

(22) Filed: Nov. 19, 2004

Related U.S. Application Data
(60) Provisional application No. 60/523,701, filed on Nov. 21, 2003.

(51) Int. Cl. A61B 19/00

(52) U.S. Cl. 604/403

(57) ABSTRACT

Disclosed herein is a system for the collection, treatment and delivery of an autologous blood sample, comprising a first syringe having a first body portion. A first sample transfer portion has a first syringe inlet for drawing an untreated blood sample from a patient and a first syringe outlet for dispensing the untreated blood sample. A blood sample treatment chamber having a chamber inlet, the first syringe outlet being operable to establish a dedicated first fluid coupling with the chamber inlet to dispense the untreated blood sample to the blood sample treatment chamber. The blood sample treatment chamber having a chamber outlet for dispensing a treated blood sample following treatment. The second syringe has a second body portion and a second sample transfer portion, the second sample transfer portion having a passage with a first access location which is operable to form a dedicated second fluid coupling with the chamber outlet. The second body portion has a second syringe outlet, the passage having a second access location for fluid communication with the second syringe outlet, releasable lock means for forming a locked third fluid coupling between the second access location and the second syringe outlet. The lock means is operable in response to a release signal to release the third fluid coupling, the second syringe outlet being operable when released from the third fluid coupling to form a fourth fluid coupling with a blood sample delivery unit.

This application claims priority under 35 U.S.C. § 119(e) with respect to U.S. provisional application No. 60/523,701 filed on Nov. 21, 2003.

### REFERENCE TO CO-PENDING APPLICATIONS

The entire subject matter of U.S. Provisional application Ser. No. 60/421,781 filed Oct. 29, 2002 and entitled DEVICE AND METHOD FOR CONTROLLED EXPRESSION OF GASES FROM MEDICAL FLUIDS DELIVERY SYSTEMS is incorporated by reference.

The entire subject matter of U.S. Provisional application Ser. No. 60/428,942 filed Nov. 26, 2002 and entitled BLOOD TREATMENT CONTROL SYSTEM is incorporated by reference.

The entire subject matter of U.S. Provisional application Ser. No. 60/464,659 filed Apr. 23, 2003 and entitled DISPENSING SYSTEMS is incorporated by reference.

The entire subject matter of U.S. Provisional application Ser. No. 60/482,725 filed Jun. 27, 2003 and entitled MEDICAL TREATMENT CONTROL SYSTEM is incorporated by reference.

The entire subject matter of PCT Patent application filed Oct. 28, 2003 under serial number PCT/CA03/01645, entitled DEVICE AND METHOD FOR CONTROLLED EXPRESSION OF GASES FROM MEDICAL FLUIDS DELIVERY SYSTEMS, and designating the United States, is incorporated by reference.

### BACKGROUND OF THE INVENTION

1. Field of the Invention

The present invention relates to the management of medical treatments.

2. Description of the Related Art

There has been, in recent years, tremendous changes in the way in which patients are treated. Most social Medicare systems have been changed to improve productivity. These changes have not occurred, however, without problems. A recent heart lung transplant surgery went horribly wrong because of a relatively minor oversight—a mismatch in the blood type of the donor and recipient patients. This event is overshadowed by accounts of patients being given the wrong medication. This suggests the need for improved monitoring of patients and their treatments to be sure they are given proper medications and/or medical procedures, given the specific, and perhaps unique, needs of each patient.

It is an object of the present invention to provide a novel medical material handling system.

### SUMMARY OF THE INVENTION

In one of its aspects, the present invention provides a system for the collection, treatment and delivery of an autologous blood sample, comprising a first syringe having a first body portion, a first sample transfer portion having a first syringe inlet for drawing an untreated blood sample from a patient and a first syringe outlet for dispensing the untreated blood sample; a blood sample treatment chamber having a chamber inlet, the first syringe outlet being operable to establish a dedicated first fluid coupling with the chamber inlet to dispense the untreated blood sample to the blood sample treatment chamber, the blood sample treatment chamber having a chamber outlet for dispensing a treated blood sample following treatment; a second syringe having a second body portion and a second sample transfer portion, the second sample transfer portion having a passage with a first access location which is operable to form a dedicated second fluid coupling with the chamber outlet, the second body portion having a second syringe outlet, the passage having a second access location for fluid communication with the second syringe outlet, releasable lock means for forming a locked third fluid coupling between the second access location and the second syringe outlet, the lock means being operable in response to a release signal to release the third fluid coupling, the second syringe outlet being operable when released from the third fluid coupling to form a fourth fluid coupling with a blood sample delivery unit.

In one embodiment, identification means is provided for identifying an originating patient for the untreated blood sample, verification means for verifying a match between the originating patient and the treated blood sample, and release signal generating means for generating a release signal in response to a positive verification by the verification means.

The identification means and/or the release signal generating means may be located on the second syringe body, on the second sample transfer portion or on an external article. The external article may worn, carried, attached or ingested by the patient, such as a pinned or self adhesive label, or a coated object, and the like. Preferably, the external article contains a removable portion containing audit data relating to the patient and/or the treated blood sample. The external article may be conveniently provided as a wrist band to be worn by the originating patient.

In one embodiment, the verification means includes comparison means for comparing originating patient identity data with the treated blood sample identity data, signal receiving means to receive one or more signals associated with, or some cases containing, the originating patient identity data and/or the blood sample identity data, and memory means for storing the patient identity data and the treated blood sample identity data. The memory means stores time value data to determine at least one time value related to a predetermined event including or between an untreated blood sample collection event and a treated blood sample delivery event.

The time value may also, if desired, include at least one elapsed time value between two predetermined events including or between the untreated blood sample collection event and the treated blood sample delivery event. In this case, the verification means may be operable to prevent release of the locked third fluid coupling when the elapsed time value has exceeded a predetermined elapsed time maximum value. The verification means may also function in association with a blood treatment unit to treat the blood sample in the blood sample treatment chamber. In this case, the verification means may be operable to prevent treatment of the blood sample when the elapsed time value has exceeded a predetermined elapsed time maximum value.

In one embodiment, the verification means is operable to verify a match between the untreated blood sample in the first syringe and the originating patient. The first syringe is assigned a first syringe identity code which is representative of the untreated blood sample therein, and the originating patient is assigned an originating patient identity code which is representative of the originating patient, wherein the first syringe and originating patient identity codes include related or common data. The first syringe identity code may also include a first time value representative of the time of untreated sample collection from the originating patient and/or verification thereof. The second syringe is also assigned a second syringe identity code, which is representative of the treated blood sample therein. The second syringe identity code may also include a second time value representative of the time of the treated sample delivery thereto from the blood sample treatment chamber and/or verification thereof.

In one embodiment, the identification means includes a first signal emitting means for emitting a first signal carrying untreated blood sample identity data and a first signal receiving means to receive the first signal. The first signal emitting means may conveniently be located on the first syringe. The identification means also includes a second signal emitting means for emitting a second signal carrying the treated blood sample identity data and a second signal receiving means to receive the second signal and the second signal emitting means may be conveniently located on the second syringe.

In one embodiment, the first body portion of the first syringe includes an untreated blood sample receiving chamber and the first sample transfer portion includes a passage joining the untreated blood sample receiving chamber with the first syringe inlet and the first syringe outlet, and first syringe inlet valve means for controlling the flow of blood through the first syringe inlet. In this case, the first syringe inlet valve means includes a septum, further comprising a complementary penetrating member located on or intermediate to the external blood collection article and in an engaged position therewith.

If desired, the second blood sample transfer portion may also include a filtered vent outlet in the passage for expelling one or more gas constituents in the treated blood sample.

In one embodiment, one or both of the dedicated first and second couplings are operable releasably to lock the first sample transfer portion of the first syringe and the second sample transfer portion of the second syringe with the blood sample treatment chamber in respective open fluid transfer conditions. In this case, the first and second couplings are configured so that the dedicated first and second couplings establish the locked open fluid transfer condition by a relative rotational displacement between the blood sample treatment chamber and the corresponding first and second sample transfer portions.

In one embodiment, the second syringe outlet includes second syringe outlet valve means for controlling the flow of the blood sample there through and which does so in two stages. In a first stage, the second syringe outlet valve means includes a valve element potion and a valve seat portion, and actuating means for actuating the valve element portion relative to the valve seat portion, the actuating means being operable to displace the valve element from the valve seat portion when the second body portion is engaged with the second sample transfer portion. The second sample transfer portion includes a housing to receive the second syringe outlet therein, the housing having a female member in fluid communication with the second access location, the second syringe outlet including a male portion to engage the female portion, the actuating means including an actuating portion adjacent the male portion to be displaced by the female portion on engagement of the female portion with the male portion. An outer sheath portion is spaced from the male portion to form an annular female portion-receiving passage therein, the actuating portion including at least one first actuating element positioned in the annular passage.

In a second stage, the actuating means is operable to engage the valve element with the valve seat portion when the second body portion is separated from the second sample transfer portion, and a second syringe outlet end portion extending outwardly from the second body portion. In this case, the actuating portion includes at least one second valve actuating element which extends laterally outwardly beyond the second syringe outlet end portion. The second outlet end portion has a bevelled distal end and the second valve actuating element has a distal end region which is configured to engage the bevelled distal end of the second outlet end portion. The distal end region of the second valve actuating element is angled to nest with the bevelled distal end of the second outlet end portion when the valve element portion is engaged with the valve seat portion. Thus, the second valve actuating element is arranged to travel along an outside surface of the second outlet end portion as the valve portion is displaced relative to the valve seat portion. A collar member is located within the housing, the collar member including a chamber to receive the second outlet end portion to form the third fluid coupling. The second valve actuating element includes an abutment flange extending outwardly therefrom, the abutment flange being operable to abut a designated location in the chamber when the second syringe outlet is removed from the chamber.

In one embodiment, the releasable lock means includes a barrier member positioned adjacent the second access location and moveable between a locked position in which the barrier member engages the second outlet end portion, and a release position in which the barrier member is retracted from the second outlet end portion. The barrier member is biased to the release position and a brace means for bracing the barrier in the locked position, wherein the brace means is releasable in the presence of a predetermined current.

In another of its aspects, the present invention provides a material dispensing device, comprising a material container portion and a material transfer portion, the material transfer portion including a passage for the transfer of materials to and from the material container portion, the passage having a first access location in fluid communication with the material container portion and a second access location, and second access location control means for controlling the flow of material through the second access location, the second access location control means including a penetrable septum which is operable in an unpenetrated condition in which the passage is closed and a penetrated condition in which the passage is open, and a third access location, the third access location including a means for forming a dedicated fluid coupling with a medical materials dispenser.

The material container portion may be is integrally formed with or separable from the material transfer portion.

In one embodiment, the septum includes a block of resilient material, having a diameter and a depth, wherein the depth approximates the diameter. A septum housing portion contains the septum and a penetrating member is provided for penetrating the septum. The penetrating member is associated with a flange which is engageable with the septum housing portion, preferably in a form which is complementary with an outer surface on the housing portion. The penetrating member may be provided in a number of forms including a hollow or grooved spike member.

In one embodiment, a locking arrangement is provided to control access to the septum. In this case, the septum is located adjacent an end flange, the end flange having an opening with a predetermined cross section to match the cross section of the spike member. The septum has an inner septum passage adjacent the end flange, and at least one lock or preferably a pair of lock members is movable between an operable position to obstruct the inner septum passage and an inoperable position, the lock member further including displacement means for displacing the locking member to the inoperable position by the presence of the spike member of a minimum lateral dimension in the inner septum passage. Each lock member has an outer lock flange and wherein, in the operable position, the lock flanges overlap one another. The displacement means includes a shank portion located on each lock member adjacent the outer lock flange, the shank portions arranged to lie adjacent one another in the inner septum passage in the operable position, the shank portions being movable to the inoperable position when the spike member of minimum lateral dimension is introduced between the shank portions.

In yet another of its aspects, there is provided a material dispensing device, comprising a chamber and an outlet, valve means for controlling the outlet, the valve means including a penetrable septum which is operable in an unpenetrated condition in which the chamber is closed and a penetrated condition in which the chamber is open, the septum including a block of resilient material having a diameter and a depth, wherein the depth approximates the diameter, an end flange, the end flange having an opening with a predetermined cross section, and a penetrating member for penetrating the septum to open the chamber, the penetrating member having a matching cross section which matches the cross section of the opening in close fitting relationship therewith, wherein the opening prevents access to the septum by penetrating members without the matching cross section.

In still another of its aspects, there is provided a material dispensing device, comprising a chamber and an outlet, valve means for controlling the outlet, the valve means including a penetrable septum which is operable in an unpenetrated condition in which the chamber is closed and a penetrated condition in which the chamber is open, the septum including a block of resilient material having a diameter and a depth, wherein the depth approximates the diameter, the septum having an inner septum passage, at least one lock member movable between an operable position to obstruct the inner septum passage and an inoperable position, the lock member further including displacement means for displacing the locking member to the inoperable position by the presence of a penetrating member of a minimum lateral dimension in the inner septum passage.

In yet another of its aspects, there is provided a device for controlling a medical materials dispenser, comprising a control portion, the control portion having a housing with a passage therein, the passage forming a first fluid coupling with a delivery outlet portion on the medical materials dispenser, and a second fluid coupling with a medical materials receptacle; and releasable locking means for locking the first fluid coupling, the lock means being operable between a locked condition and unlocked condition in response to an actuation signal generated by an external device.

In one embodiment, the passage includes a gas discharge vent to permit the gas from the medical materials dispenser to be discharged there through. The releasable lock means includes a barrier member moveable between a locked position in which the barrier member engages the delivery outlet portion and a release position in which the barrier member is retracted from the delivery outlet portion. Desirably, the barrier member is biased to the release position. In addition, brace means for bracing the barrier in the locked position, wherein the brace means is releasable in the presence of a predetermined current.

In still another of its aspects, there is provided a syringe device comprising a syringe body, the syringe body having a first body portion with a cavity formed therein, a plunger in sealed engagement with the cavity to form a fluid receiving chamber, the syringe body having a second body portion, the second body portion having a passage formed therein, the passage having a first access location in fluid communication with the chamber and a second end terminating at a second access location, the passage having a third access location, wherein at least one of the second and third access locations includes a penetrable septum which is operable in an unpenetrated condition in which the passage is closed and in a penetrated condition in which the passage is open.

As a further aspect, the present invention provides a method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
collecting the sample in a second collection device;
associating the second collection device with a third signal carrying data representative of the processed sample;
comparing the data in the first and third signals to link the processed sample with the patient; and thereafter;
permitting the processed sample to be released after a positive association of the data.

As a further aspect, the present invention provides a method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
collecting the processed sample in a second collection device, wherein the second collection device is locked against release of the processed sample in the absence of an unlocking signal;
associating the second collection device with a third signal carrying data representative of the processed sample;
comparing the data in the first and third signals to link the processed sample with the patient; and thereafter;
unlocking the second collection device to permit release of the processed sample when an unlocking signal is sent after a positive association has been made between the processed sample and the patient.

As yet a further aspect, the present invention provides method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
associating the processed sample with a third signal carrying data representative of the processed sample;
comparing the data in the first and third signals to link the sample as processed with the patient and thereafter;
assembling a patient record including the data in one or more of the first, second and third signals.

The term “treatment device” used herein below is intended to mean a device used directly or indirectly in the course of a treatment. It may include devices which actually perform a treatment on the patient or a patient-derived sample, or alternatively be an article for performing functions associated with treatments, such as carrying or otherwise transferring the sample to or from a treatment. Several other examples of such treatment devices are described herein.

### BRIEF DESCRIPTION OF THE DRAWINGS

Several preferred embodiments of the present invention will now be described, by way of example only, with reference to the appended drawings in which:

FIG. 1 is a perspective view of a blood treatment system;

FIG. 2 is a sectional view of a first syringe shown in FIG. 1;

FIG. 3 is a magnified fragmentary sectional view of an end region of the first syringe of FIG. 2;

FIGS. 4a, 4b and 4c are fragmentary sectional views of an alternative end region to that shown in FIG. 3;

FIG. 5 is a fragmentary perspective view of a portion of locking arrangement in the end region of FIGS. 4a, 4b and 4c;

FIG. 6 is a perspective view of a component used with the first syringe of FIG. 2;

FIG. 7 is a sequential view of an operation using the first syringe of FIG. 2;

FIG. 8 is a fragmentary perspective view of a sample treatment chamber of the system of FIG. 1;

FIGS. 9 and 9a are fragmentary sectional views of the sample treatment chamber in FIG. 8;

FIGS. 10 and 11 are fragmentary sectional views of the sample treatment chamber in FIG. 8 in an operative position with the first syringe of FIG. 1 and a second syringe, also of FIG. 1;

FIG. 12 is a partial exploded view of the first and second syringes of FIG. 1, together with a portion of the sample treatment chamber of FIG. 8;

FIG. 13 is a perspective sequential view showing installation of the first and second syringes on the treatment chamber of FIG. 8;

FIGS. 14, 15 and 16 are perspective and fragmentary sectional perspective views respectively of the second syringe of FIG. 1;

FIG. 17 is perspective view showing alternative assemblies for the second syringe;

FIGS. 18 to 23 are fragmentary perspective views of the second syringe or portions thereof

FIGS. 24a, 24b and 25 are fragmentary sectional and perspective views, respectively, of a portion of the second syringe;

FIGS. 25a to 25d show an alternative to one component of the second syringe shown in FIG. 25;

FIGS. 26 and 27 are schematic views of a verification portion of the system of FIG. 1;

FIGS. 28 and 29 are schematic views of a verification protocol; and

FIG. 30 positions to 32 are perspective views of a wrist band as shown in FIG. 1, in different operative

### DESCRIPTION OF THE PREFERRED EMBODIMENTS

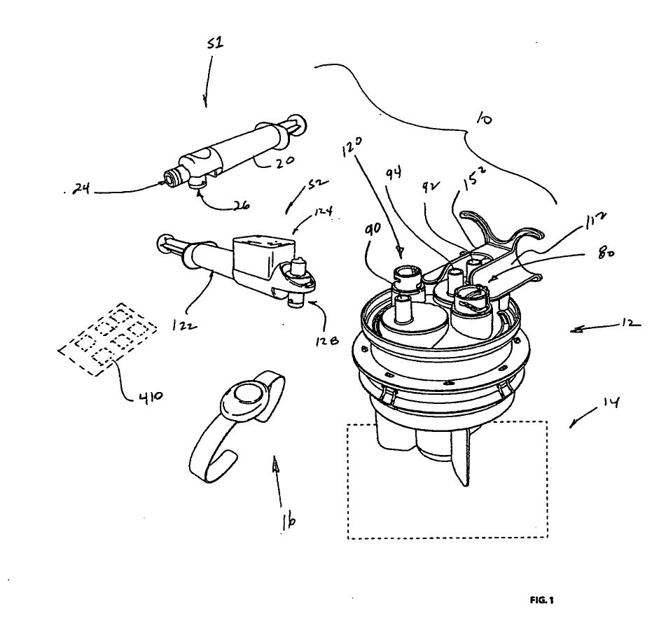

Referring to the figures, particularly FIG. 1, there is provided a system 10 for the collection, treatment and delivery of an autologous blood sample. The system 10 has a number of components which are used at different stages during the handling of the blood sample. As will be described, the system makes use of a first blood sample collection syringe S1 which used to collect an untreated blood sample from an originating patient. Following blood sample collection, the first syringe S1 is connected to a blood treatment chamber 12 which is then delivered to a blood treatment unit shown schematically at 14 in which the blood sample is subjected to one or more stressors as, for example, described in PCT application serial number PCT/CAOO/0 1078 filed Sep. 15, 2000 entitled APPARATUS AND PROCESS FOR CONDITIONING MAMMALIAN BLOOD (the entire contents of which are incorporated herein by reference).

Following treatment, the treated blood sample is delivered to a second syringe S2 which then is used to deliver the treated sample to the originating patient. At one or more critical stages, the system 10 provides for a verification check, aimed at reducing the possibility of error, to ensure that the correct blood sample is returned to the correct originating patient. This is done by matching the blood sample, either in its treated or untreated form or both, with the originating patient by comparing originating patient identity data and sample identity data. To that end, the system 10 is provided with a patient wrist band 16 which is capable of communicating with controlling or communication functions in the S1 syringe and/or the S2 syringe to aid in this verification.

Even though the description below is in large part focused on the use of system 10 in the treatment of autologous blood samples, it will be understood that the system, its components and alternatives thereof, may be used for autologous samples other than blood samples, such as bone marrow or, lymphatic fluids, semen, ova-fluid mixtures, other bodily fluids or other medical fluids which may or may not be “autologous”, for example fluid mixtures perhaps containing a patient desired solid sample such as from organs, body cells and cell tissue, skin cells and skin samples, spinal cords. The system may also be used for medical testing where it is important to ensure that test results of a particular test can be delivered to the originating patient.

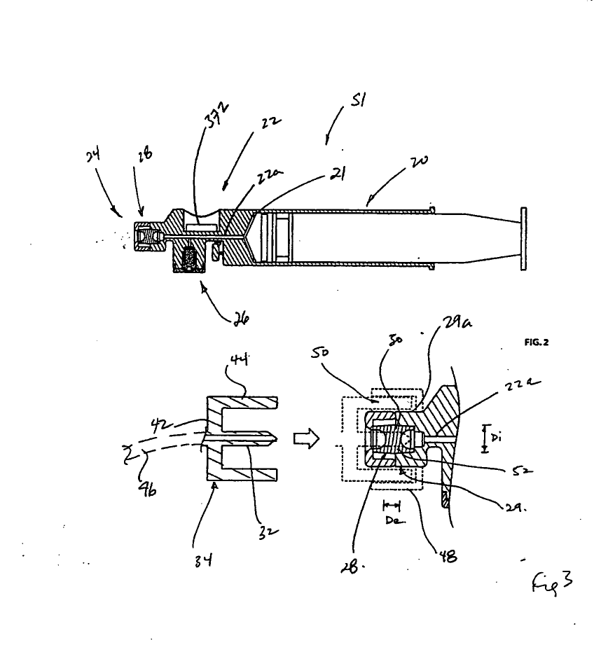

Referring to FIGS. 1 and 2, the first syringe S1 has a first body portion 20 which provides a cylindrical cavity which in cooperation with a syringe plunger forms a sample receiving chamber 21. The first syringe is also provided with a channel portion 22 which provides a channel 22a joining the sample receiving chamber 21 with a first syringe inlet 24 for drawing an untreated blood sample from a patient and a first syringe outlet 26 for dispensing the untreated blood sample therefrom to the blood sample treatment chamber 12.

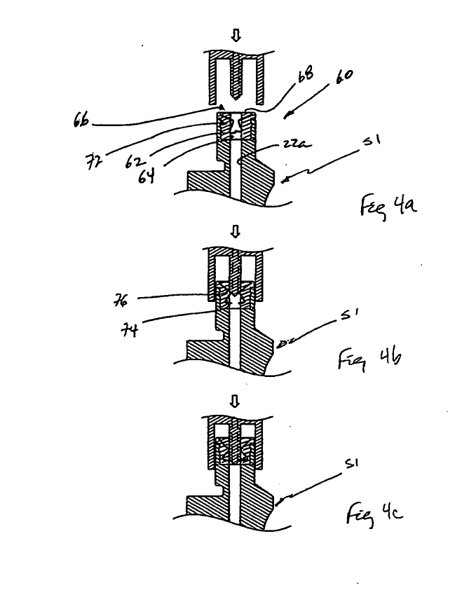

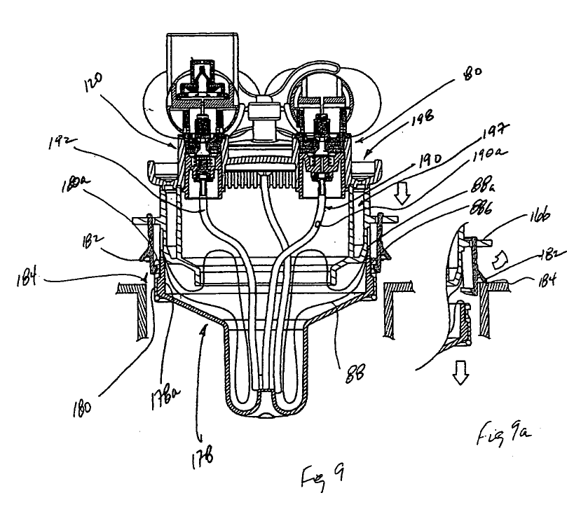

Referring to FIGS. 2 and 3 the first syringe inlet 24 of the first syringe S1 is provided with a first syringe inlet valve means 28 for controlling the flow of blood through the first syringe inlet 24. In this case, the first inlet valve means 28 includes housing 29 containing a septum 30 which is arranged to be opened by a complementary penetrating member shown at 32, located on or intermediate to an external device 34. The external device may be a blood collection unit 34 in FIG. 6 (in the form referred to as a “butterfly”) or an adaptor 36 to join to a vial 38 or the like as shown in FIG. 7. In this case, the adaptor 36 has a pair of opposed spikes 32, 40, one to penetrate the septum on the syringe S1 and the other to penetrate a septum on the vial 38.

Referring to FIG. 3, the blood collection device 34 includes a base 42 supporting the penetrating member 32 and a flange 44, which is complementary and engageable with an outer surface 29a of the housing portion 29. In this case, the penetrating member 32 is in the form of a hollow spike and a conduit 46 is positioned opposite the spike and in fluid communication therewith. The spike may also be in number of other forms, such as spike with an external groove or channel. The housing portion 29 may also be provided with an outer web 48 shown in phantom cross-section in FIG. 3, which is spaced from the outer surface 29a to form a peripheral cavity 50 to receive the flange 44 on the blood collection unit 34.

Referring to FIG. 3, the septum 30 is provided in the form of a block of resilient material 52 and has a diameter Di and a depth De, wherein the depth De approximates the diameter Di. It will be seen that, when the spike 32 is in the engaged (or penetrating) position with the septum 30, the blood sample (or other fluid material) can now flow through the spike 32. In this case, the spike 32 has a depth which is equal to or greater than the depth of the septum. However, there may also cases where the spike 32 has a length that is smaller than the depth of the septum provided that a satisfactory fluid communication is established between the spike 32 and the channel 22a.

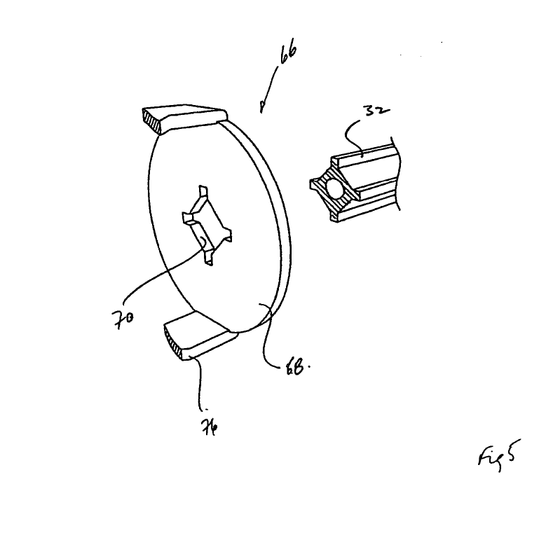

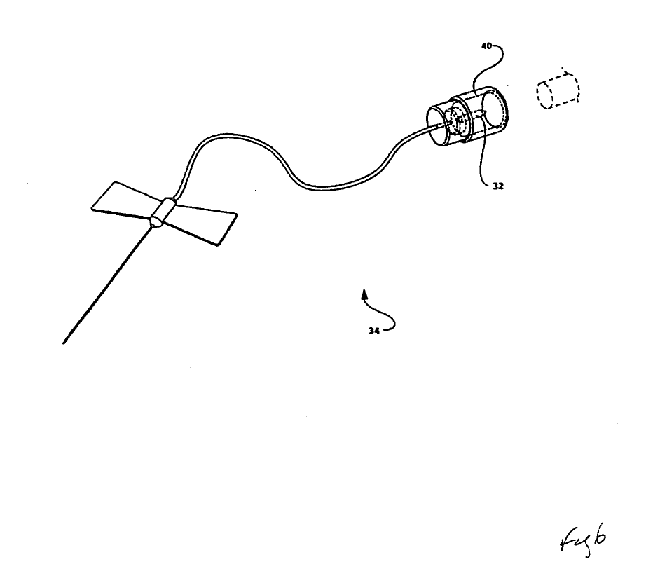

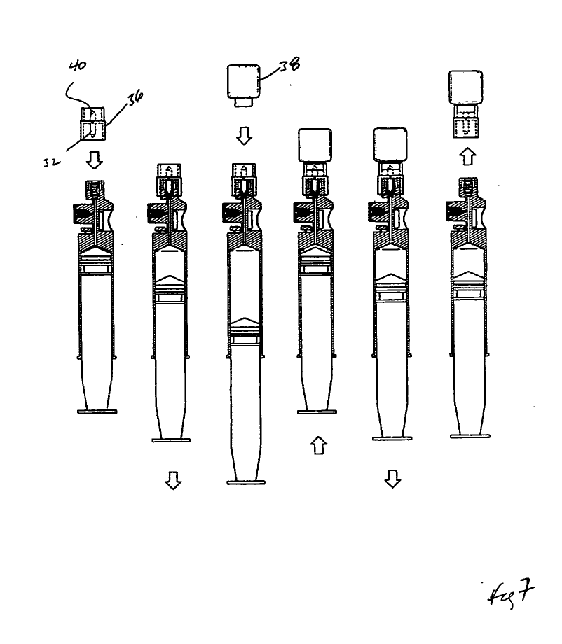

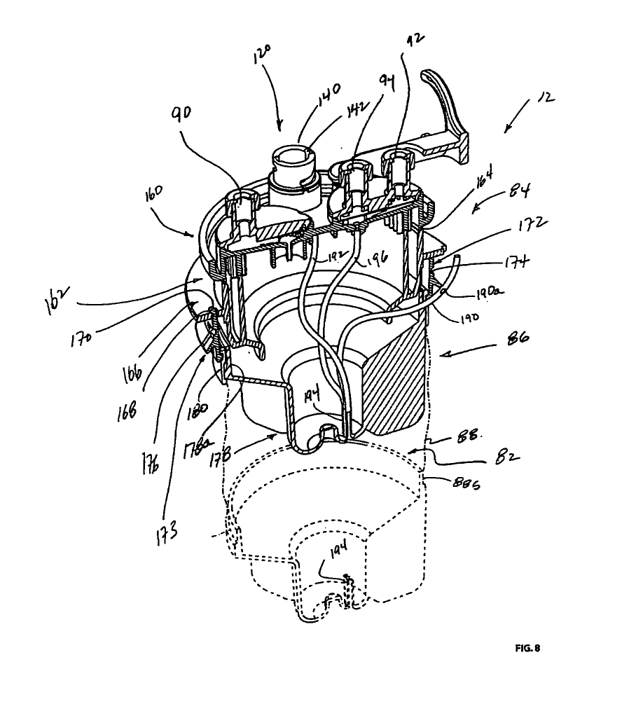

FIGS. 4a to 4c and FIG. 5 illustrate another first syringe inlet 60 on an alternative syringe S1 having a resilient blank member forming a septum 62 with an inner septum passage 64, which is in fluid communication with the channel 22a. Located within the septum 62 is a locking assembly 66 for controlling access to the septum channel 62 to only those spike members having the required cross section and lateral dimensions. Referring to FIG. 5, the locking assembly 66 has an end flange 68 with an opening 70 which has a predetermined cross section to match the cross section of the spike 32. It follows that the end flange 68 may be configured uniquely with one or more spikes 32, much in the same manner as a lock and key. This arrangement prevents unauthorized spikes, that is with unapproved cross sections, from being used with the septum 62, thus providing a first level of security.

The inner septum passage 64 is aligned with the opening 70 of end flange 68. As a second level of security, the locking assembly 66 is equipped with a pair of overlapping lock members 72 which are movable between an operable position to obstruct the inner septum passage 64 and an inoperable position. As will be described, the locking assembly 66 further includes displacement means for displacing the locking assembly to the inoperable position by the presence of the spike of a minimum lateral dimension in the inner septum passage 64.

Each of the lock members 72 includes an outer lock flange 74 which overlap one another in the operable position. The displacement means includes a shank portion 76 located on each lock member 72 adjacent the outer lock flange 74 and are integrally formed with the end flange 68. The shank portions 76 lie adjacent one another in the inner septum passage 64 in the operable position and are movable to the inoperable position when the spike of minimum lateral dimension is introduced between the shank portions 76.

Thus, if there is an attempt to access the septum passage 64 with an unauthorized small object such as a needle that would otherwise fit through the opening 70, the width of the unauthorized small object will be insufficient to engage and laterally outwardly displace the shank portions 76. As a result, the over lapping lock members 74 remain as barriers to the septum passage 64 upstream thereof. On the other hand, a properly sized spike engages the shank portions 76 and displaces them a sufficient distance to bring the lock members 72 out of the overlap, thereby opening the septum passage 64.

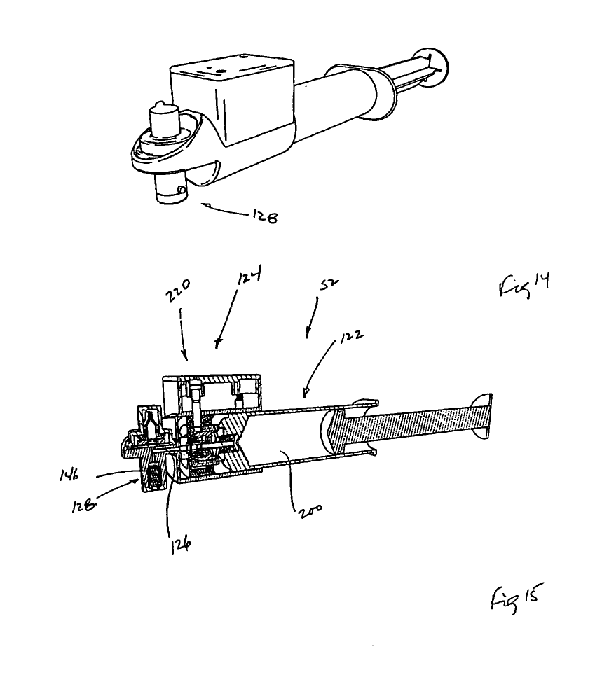

Referring to FIGS. 1 and 8 to 12, the blood sample treatment chamber 12 has a chamber inlet 80 to form a dedicated first fluid coupling with the first syringe outlet 26, in order that the untreated blood sample may be dispensed to the blood sample treatment chamber 12. The blood sample treatment chamber 12 has an expandable treatment cavity 82 formed by a cover portion 84, a bottom portion 86 and a flexible walled portion there between as shown at 88. The chamber 12 also has a gas inlet port 90 for delivery of ozone or other stressors to treat a blood sample, a gas outlet port 92 for the discharge of the ozone, and an expansion gas exchange port 94, which provides a source of pressure to expand (or vacuum to retract) the chamber before (or after) treatment. Other features of the treatment chamber can be found in copending U.S. Provisional application Ser. No. 60/482,725 filed Jun. 27, 2003 and entitled MEDICAL TREATMENT CONTROL SYSTEM.

The flexible walled portion is made from low density polyethylene (LDPE) containing a small amount (about 5%) to of ethylene vinyl acetate and is capable of transmitting radiation in the UVA, B and C as well as infrared ranges. The other components of the treatment chamber 12 should also be similarly transmissive of radiation of these wavelengths, and particularly the bowl 178 which will receive the blood sample during treatment.

The chamber inlet 80 has a female collar portion 100 with a pair of helically oriented passages or grooves 102 extending through or in its wall to engage a corresponding one or more pins 104 extending outwardly from the first syringe outlet 26.

A valve element 106 is located in the channel 22a of syringe SI and biased to a closed position against a valve seat 108 on an end cap 109 which forms the outer end of the first syringe outlet 26. The valve element 106 is aligned for abutment with a valve actuating element 110 which is positioned in the chamber inlet 80. The valve actuating element 110 is operable to displace the valve element 106 from its closed position against the valve seat 108 to open the fluid coupling.

The syringe S1 is thus interconnected to the chamber inlet 80 by aligning the first syringe outlet 26 with the female collar portion 100 so that the pins 104 engage the helical passages 102. The syringe S1 is then rotated in the manner shown in FIG. 13, thus carrying the pins 104 to progress along the helical passages 102 and downwardly into the female collar portion until such time as the valve element 106 is urged open by its abutment with the valve actuating element 110. The treatment chamber 12 is also provided with a saddle member 112 for supporting the syringe S1 in the fully engaged position with chamber inlet 20.

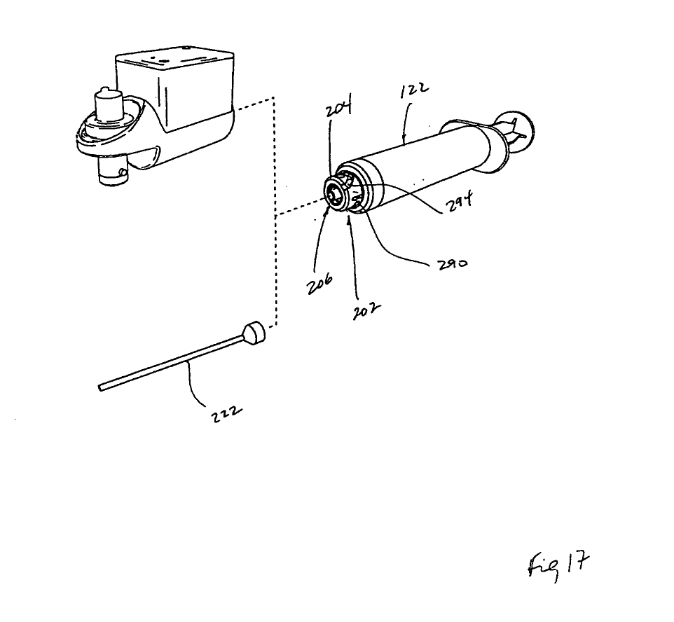

The blood sample treatment chamber 12 has a chamber outlet 120 to form a dedicated second fluid coupling with the second syringe 52. Referring to FIGS. 1, 14 and 15, the syringe S2 has a second syringe body portion 122 and a blood sample transfer portion 124. The blood sample transfer portion 124 has a passage 126 with a first access location 128 which is operable to form the dedicated second fluid coupling only with the chamber outlet 120 in order that the blood sample, once treated, may be dispensed to the second syringe S2 for later delivery to the originating patient.

Referring once again to FIGS. 1, 8, 11 and 12, the chamber outlet 120 has a female collar portion 140 with a pair of helically oriented passages or grooves 142 extending through or in its wall to engage a corresponding one or more pins 144 extending outwardly from the second syringe outlet 128. Similarly, a valve element 146 is located in the channel 126 and biased to a closed position against a valve seat 148 on an end cap 149 forming the outer end of the second syringe outlet 128. The valve element 146 is also aligned for abutment with a valve actuating element 150 which is positioned in the chamber outlet 120. The valve actuating element 150 is thus operable to displace the valve element 146 from its closed position against the valve seat 148 to open the second fluid coupling. The treatment chamber 12 is also provided with a saddle member 152 for supporting the syringe S2 in the fully engaged position with chamber inlet 120.

Referring to FIGS. 8, 9 and 9a, the cover portion 84 has a cap member 160 and a body member 162 bonded, welded or otherwise fixed thereto at intersection 164. The body member 162 has a flange 166 extending outwardly therefrom with a number of locking passages 168, each to receive an upwardly directed tab 170 on a locking skirt 172, as viewed in FIG. 8. The skirt 172 has a number of locking flanges 173, each of which is formed by a local line of weakness 174 in the skirt and a pair of neighbouring vertical slots, one of which is shown at 176. The lower end of each locking flange 173 latches on an upper periphery of the bottom portion 86.

The bottom portion 86 has a bowl 178 to receive the blood sample, and an outer wall 178a extending outwardly therefrom. A ring 180 engages an upright portion of the outer wall 178a and has a circumferential bead 180a (FIG. 9) which is dimensioned to form a seat for the lower ends of the locking flanges 173.

As seen in FIG. 9a, the locking flanges 173 are movable outwardly relative to the flange 166. Each locking flange 173 has a catch 182 which extends outwardly to abut a portion of a channel wall 184 into which the blood sample treatment chamber 12 is placed in the sample treatment unit 14. Thus, as the treatment chamber is lowered into the treatment unit 14, contact between the locking flange 173 and the channel wall portion 184 causes the bottom portion 86 to be released from the locking skirt 172.

The chamber inlet 80 and the chamber outlet 120 are each in fluid communication with the inner treatment cavity 82 by way of conduits 190, 192 extending below the valve actuating elements 110, 150 respectively. The conduit 190 is anchored to an upright post 194 formed on an inner surface of the bottom portion 86. The conduit 190 has an opening 190a in its side wall a relatively short distance from its upper end, which allows for the blood sample in the conduit 190 to pass through the opening 190a and travel to the bottom of the inner treatment cavity 82. This minimizes blood sample from being trapped in the conduit 190 after treatment. A third conduit 196 is provided for fluid communication with both the gas outlet port 92 and the expansion gas exchange port 94.

Referring to FIG. 9, the flexible walled portion 88 is cylindrical in shape and has an upper periphery 88a which is bonded to the body member 162 and a lower periphery 88b which is bonded to the bottom portion 86 between the outer wall 178a and the ring 180. The flexible walled portion is then folded into the unexpanded treatment chamber as shown in FIG. 9.

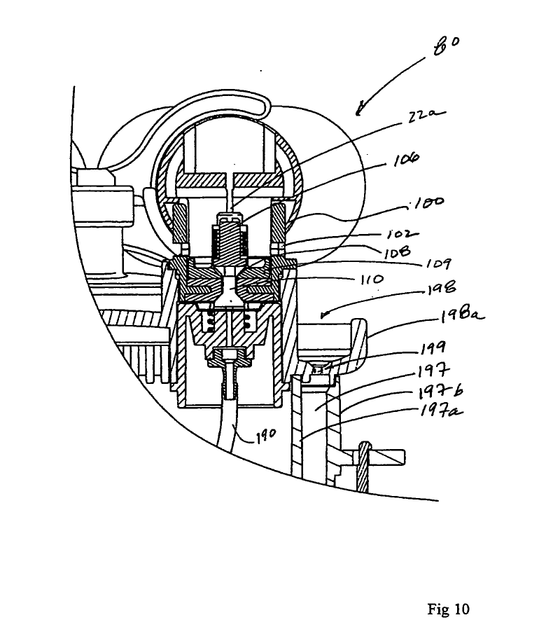

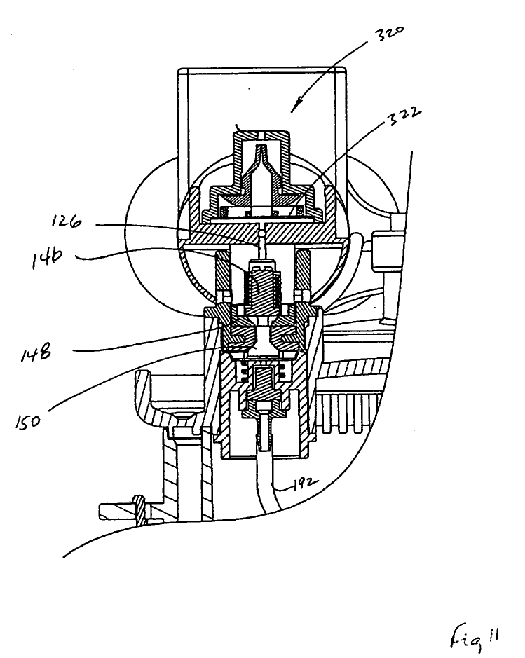

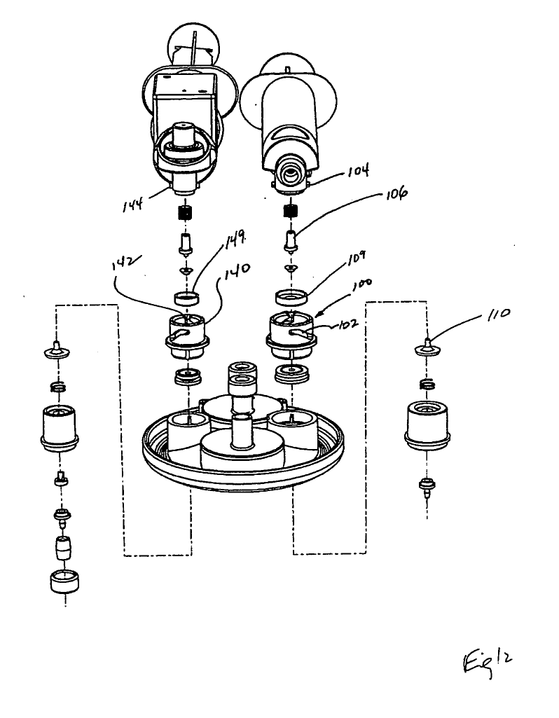

Referring to FIGS. 9 and 10, the cap member 160 is also provided with a spill collection chamber 197 bounded by inner and outer walls 197a and 197b. Located above the spill collection chamber on the cap 160 is a spill collection channel 198 outwardly bounded by the outer wall 198a. The spill collection channel 198 and the spill collection chamber 197 are joined by a number of regularly spaced passages 199. Thus, should any blood spill as a result of the coupling of either the first or second syringes with the chamber inlet and outlet, the blood will collect in the spill collection chamber.

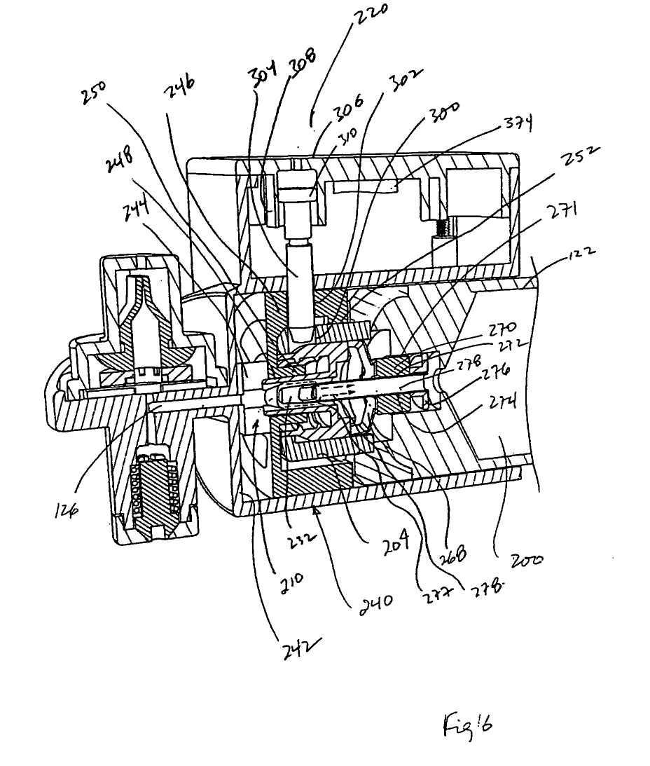

Referring to FIGS. 14 to 17, the second syringe body portion 122 has a cylindrical cavity which in cooperation with a plunger provides a sample receiving chamber 200. The second syringe body portion 122 has a second syringe outlet 202 having an outer sleeve portion 204 encircling an inner male portion 206. The passage 126 of the blood sample transfer portion 124 has a second access location 210 for fluid communication with the second syringe outlet 202.

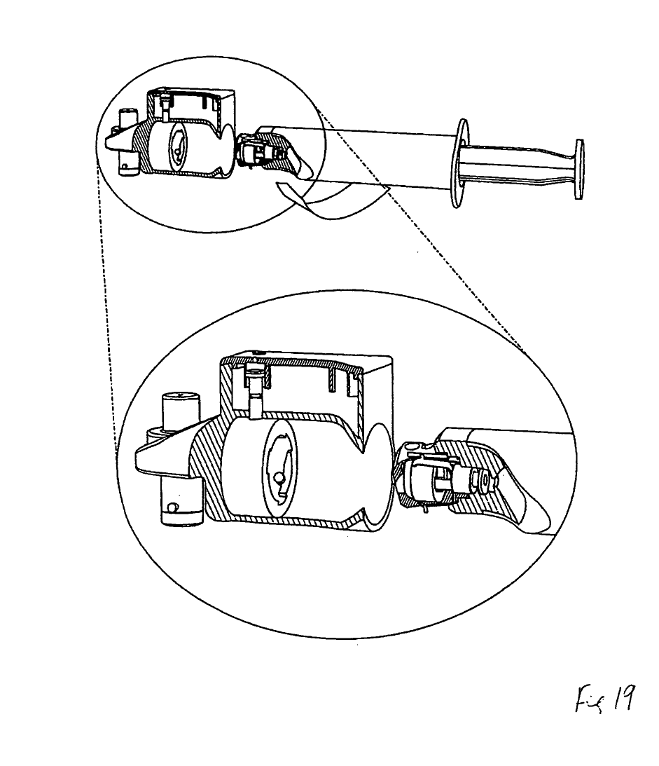

The second syringe outlet 202 and the blood transfer portion 124 are further provided with releasable lock means shown generally at 220 for forming a locked third fluid coupling between the second access location 210 and the second syringe outlet 202. As will be described, the releasable lock means 220 is operable in response to a release signal to release the third fluid coupling. When so released, the second syringe outlet 202 is operable to form a fourth fluid coupling with a fluid fitting on a common blood sample delivery unit with a complementary LUER or similar fitting, such as the needle 222 as shown in FIG. 17.

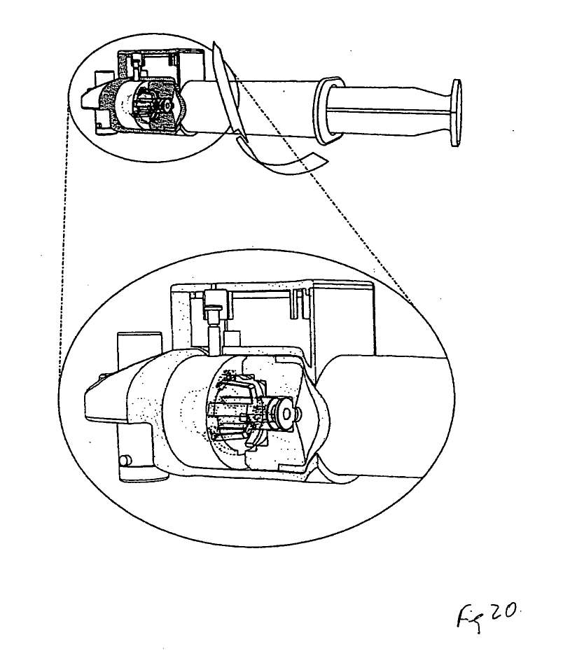

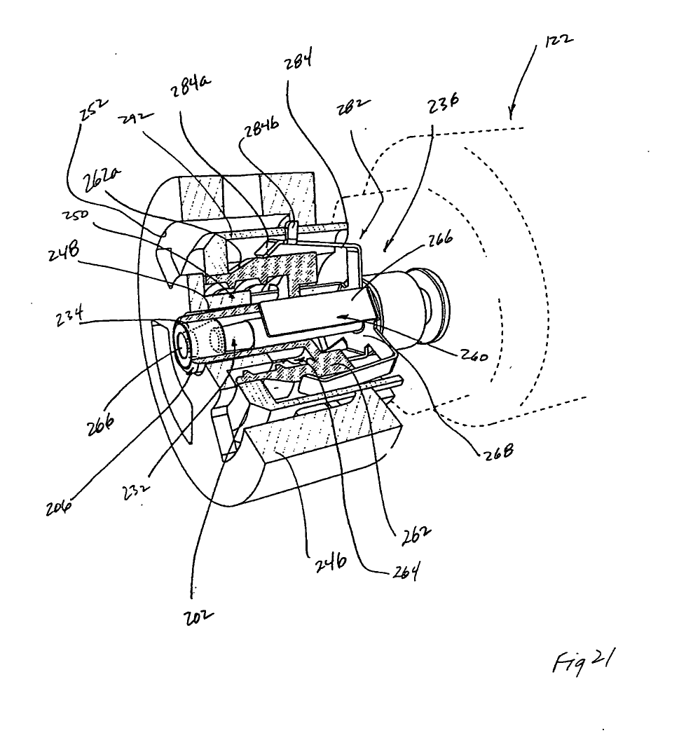

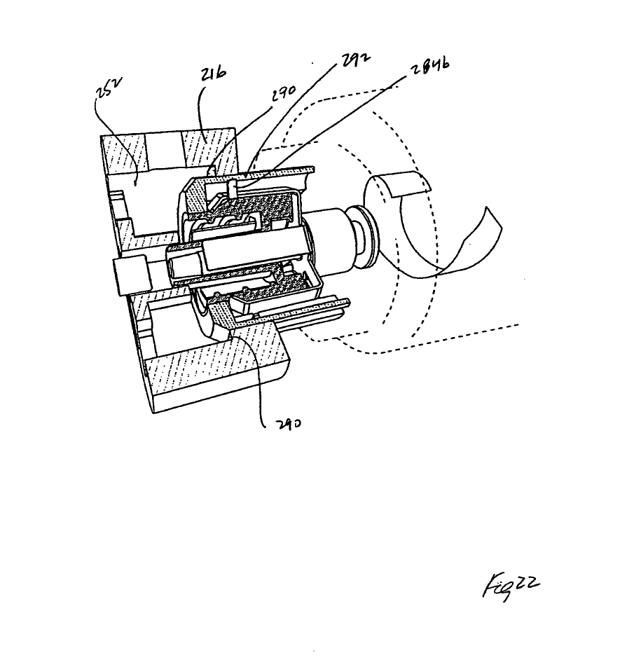

Referring to FIG. 18, the second syringe outlet 202 includes second syringe outlet valve means generally shown at 230 for controlling the flow of the blood sample there through. Referring to FIGS. 21, 22, the second syringe outlet valve means 230 includes a valve element portion 232 and a valve seat portion 234, and actuating means generally shown at 236 for actuating the valve element portion 232 relative to the valve seat portion 234. As will be described, the actuating means 236 is operable to displace the valve element portion 232 in different directions when the second syringe body portion 122 is either engaged or disengaged with the blood sample transfer portion 124.

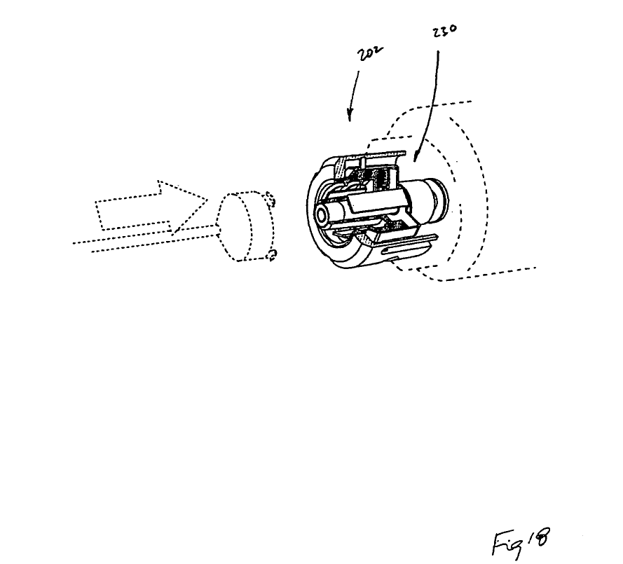

Referring to FIGS. 16 and 21, the blood sample transfer portion 124 provides a housing 240 which receives the second syringe outlet 202. The housing 240 has an inner wall 242 exposing the channel 126 and which itself terminates at a resilient seal 244. Positioned in the housing 240 against the seal 244 is a collar member 246 which has a central passage 248 which is bordered by a female member 250. The collar member 246 also has a central chamber 252 to receive the outer sleeve portion 204.

Referring to FIG. 21, the actuating means 236 includes a first actuating portion 260 adjacent the male portion 206 and which is displaced by the female member 250 when the second syringe outlet 202 is operably positioned within the collar member 246. A threaded outer sheath 262 is provided at the second syringe outlet 202 to provide a thread for the LUER fitting for coupling with the needle 222. The sheath 262 is spaced from the male portion 206 to form an annular female portion-receiving passage 264 therein. The first actuating portion 260 takes the form of a plurality of first actuating elements 266 which extend outwardly from a central web 268 and are positioned in the annular passage 264. As best seen in FIG. 16, the central web 268 is fixed to a block 270 slidably positioned in a passage 271 in the body portion 122 of the syringe S2. The block 270 has a central bore 272 carrying a tubular valve stem 274 having one end carrying the valve element portion 232 and an opposite end carrying a valve stem head 276, which has a peripheral edge region with a sealing element such as an O-ring or the like. The valve stem has a pair of fluid transfer holes as shown at 277 immediately beside the valve member portion 232, thereby forming an inner valve passage as shown in dashed lines at 278 which is in fluid communication with the chamber 200.

Accordingly, when entering the passage 264, the female member 250 makes contact and displaces the first actuating elements 266, which in turn displaces the valve stem 272 and the valve element portion 232, thus opening the inner valve passage 278 within the valve stem 272 to the second channel 126.

As best seen in FIGS. 21 and 22, the actuating means 236 includes a second actuating portion 282 having a plurality of second valve actuating elements 284 extending laterally outwardly beyond the periphery of the sheath 262. As will be described, in contrast to the function of the first valve actuating elements 266 to “open” the second outlet valve means 230, the second valve actuating elements 284 control the “closing” of the second outlet valve means 230 as the second syringe S2 is separated from the blood sample transfer portion 124.

The sheath 262 has a bevelled distal end region 262a and each of the second valve actuating elements 284 has an inwardly angled free distal end region 284a which nest with the distal end region 262a when the valve element portion 232 is engaged with the valve seat portion 234 and provides a firm yet releasable means of holding the valve element portion 232 in position against the valve seat portion 234 when the syringe S2 is removed from the housing 240. Thus, in use, the second valve actuating elements 284 travel along an outside surface of the sheath 262 as the valve element portion 232 is displaced relative to the valve seat portion 234.

The second valve actuating element 284 includes an abutment flange 284b extending outwardly therefrom which is operable to ride against an annular ridge 290 on the collar portion 246. Referring to FIG. 22, the annular ridge 290 can be seen to take an angular or helical path along the circumference of the central chamber 252. The abutment flange can be seen extending along a longitudinal slot 292 in the second syringe outlet 202 as seen in FIGS. 17, 21 and 22. The abutment flange 284b is also dimensioned so that it projects outwardly beyond the slot 292 when the valve member portion 232 is spaced from the valve seat portion (as seen in FIG. 21) but below the outer elevation of the slot 292 when the valve member portion is engaged with the valve seat portion 234 (as seen in FIG. 22). This allows for the additional user-initiated function to close the second syringe outlet valve means 230 when, for example, the syringe S2 is separated from a needle 222. In this case, once the needle 222 has been removed, the user may grip the emerging abutment flange 284 and draw the flange along the slot 292 to close the second syringe outlet valve means 230.

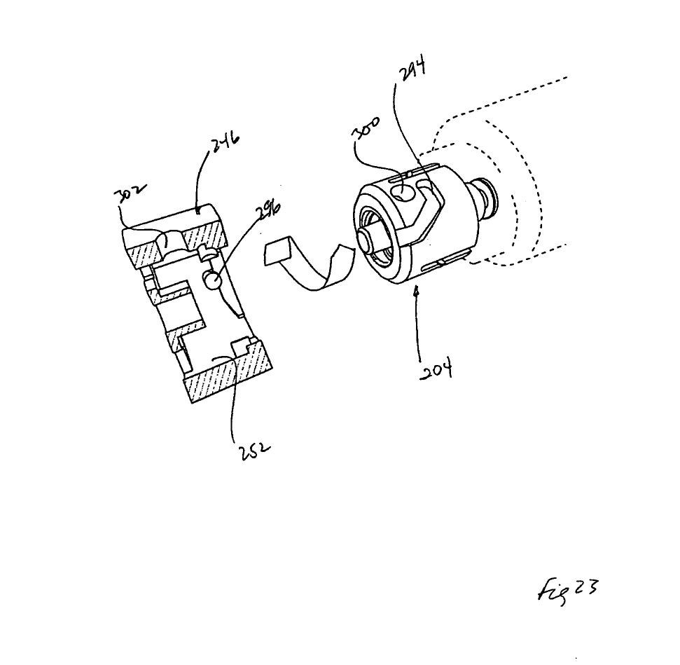

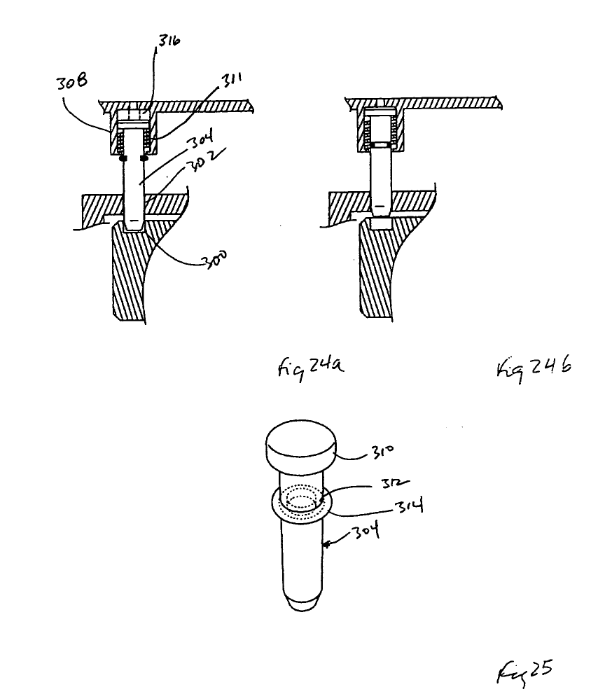

Referring to FIG. 23, the second syringe outlet 202 is provided with one or more grooves 294 which extend in a part helical fashion along the outer sleeve portion 204. The grooves 294 align with pins 296 extending into the central chamber 252 of the collar member 246, thus requiring the syringe S2 to be twisted and pulled simultaneously (as shown by the ribbon arrow in FIG. 23) to withdraw the syringe S2 from the housing 240. The outer sleeve portion 204 is also provided with a bore 300 which, when the syringe is the operatively positioned in the housing 240, aligns with a bore 302 in the collar member 246. The aligned bores 300 and 302 may be seen in FIG. 16 and are provided as part of the locking means 220, namely by receiving a locking pin 304 therein. The locking pin 304 is mounted in a lock housing 306 having a sleeve 308 carrying a head portion 310 of the pin 304 in sliding relation therewith.

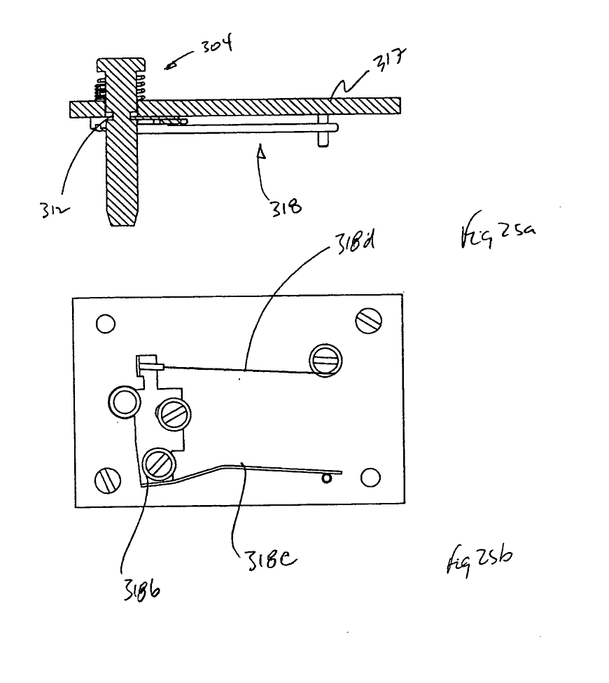

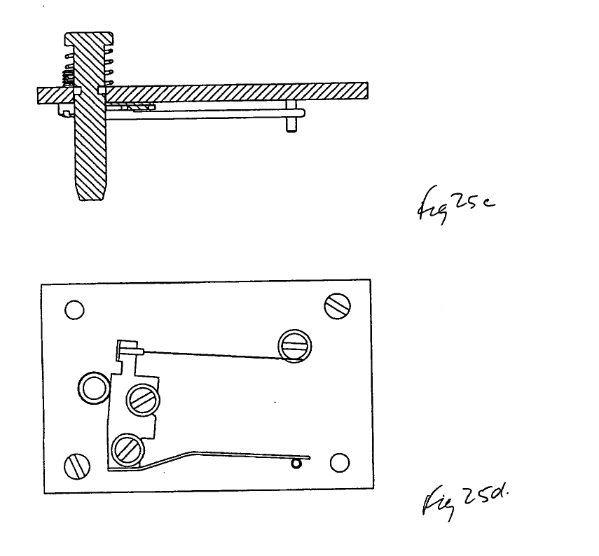

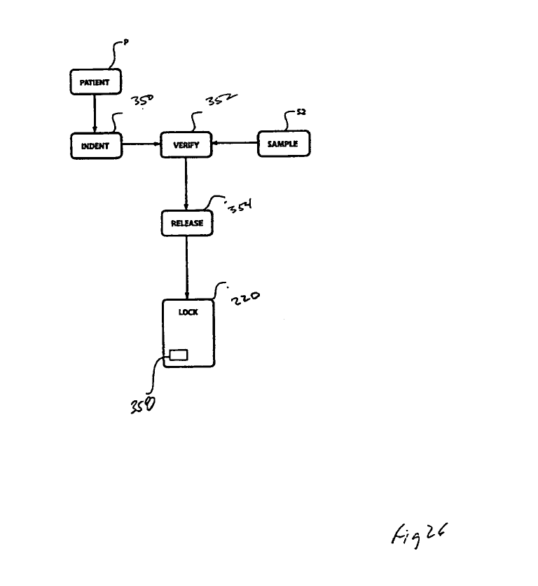

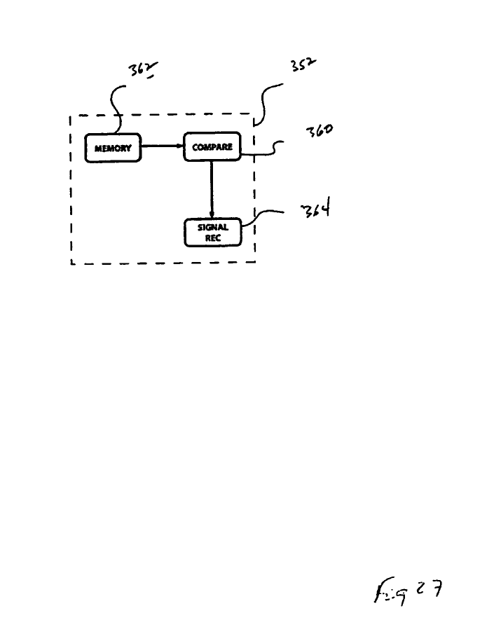

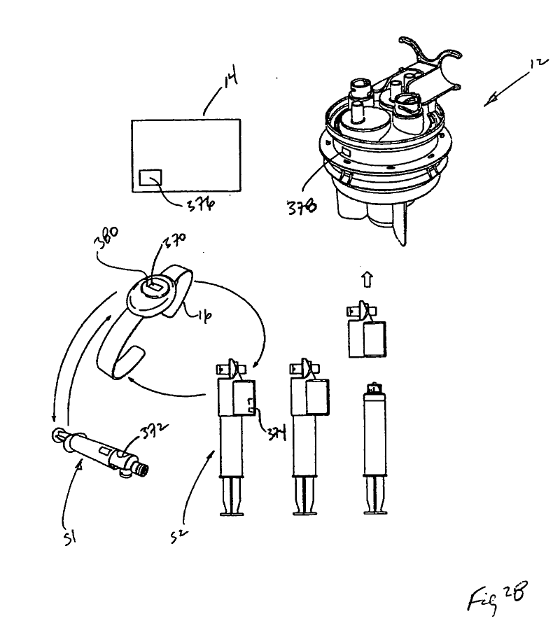

Further details of the releasable locking means 220 may be seen in FIGS. 24a, 24b and 25. A spring 311 biases the head portion 310 into the sleeve 308, thereby toward a released position where the locking pin 304 is removed from the bore 300. The locking pin 304 has a groove 312 carrying a ring 314 which is sensitive to the presence of an electric current and in the presence of which will contract from a first diameter (as shown in solid lines in FIG. 25) to a second reduced diameter, shown in dashed lines in FIG. 25. In its first diameter, the ring 314 acts as a brace to hold the locking pin in its fully extended position in bores 300, 302. Once the ring 314 receives a predetermined electric current, and thus undergoes a reducing in its diameter, the ring 314 no longer of sufficient width to brace the locking pin 304 against the sleeve 308. Consequently, spring 311 biases the locking pin 304 to a position deeper into the sleeve, thus releasing the locking pin 304 from the bore 300, and thus enabling the syringe S2 to be withdrawn from the housing 240.

There are other arrangements which can provide a similar releasable locking function by the use of a release signal. For example, the ring 314 may be replaced by a ring or loop made of fuse material which vaporizes or changes consistency under the presence of the predetermined electric current, as for example can be done with an electrical fuse material known as NITINOL. Alternatively, the ring 314 may be replaced by a post (shown in dashed lines at 316) of the same fuse material, located between the head portion 310 of the locking pin 304 and the back wall of the sleeve 308. Thus, the post 316 is operable temporarily to brace the locking pin 304 in its fully extended position. In this case, the post 316 may be provided the predetermined electric current, causing the post to vaporize and allowing the pin to be released.

An alternative arrangement is shown in FIGS. 25a to 25d, wherein the locking pin 304 is held in a plate 317 and the a spring loaded latch mechanism 318 is mounted on the plate 317 and has a latch member 318a which is pivoted on pivot member 318b and latched, in one operative position, in the groove 312, against the spring bias of spring element 318c. In this case, the fuse material is provided in the form of a trigger element 31 S1 which contracts in the presence of a predetermined current or heat and releases the latch, allowing the pin 304 to release under the action of spring 311.

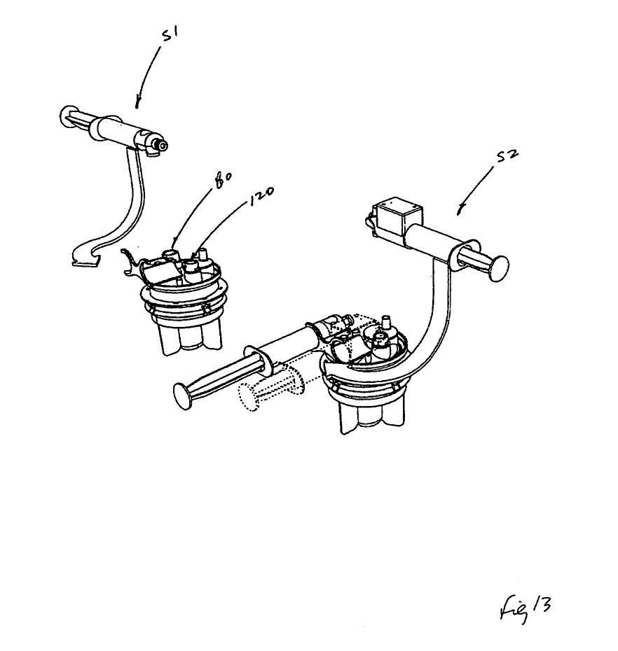

Referring to FIG. 11, the blood sample transfer portion 124 of the second syringe S2 includes a filtered vent outlet 320 in the passage 126 for expelling one or more gas constituents in the treated blood sample. In this case, the vent outlet 320 includes a barrier layer 322 which allows gaseous constituents in the blood sample to be expressed from the syringe S2 while retaining the treated sample therein. Further details of the filtered vent outlet 320 can be found in U.S. Provisional application Ser. No. 60/421,781 filed Oct. 29, 2002 and entitled DEVICE AND METHOD FOR CONTROLLED EXPRESSION OF GASES FROM MEDICAL FLUIDS DELIVERY SYSTEMS and in PCT application filed Oct. 28, 2003 under serial number PCT/CA03/01645 and entitled DEVICE AND METHOD FOR CONTROLLED EXPRESSION OF GASES FROM MEDICAL FLUIDS DELIVERY SYSTEMS.

While the system 10 makes use of syringes 51 and S2, it will be understood that other devices may be used such as, alone or in combination, one or more syringes, IV bottles, powder and/or atomized fluids and/or gas inhalant dispensers, implant delivery dispensers, ventilators, syringe pumps, intubation tubes, gastrointestinal feeding tubes, or a plurality and/or a combination thereof. One of the treatment devices may also comprise a blood treatment device such as that disclosed in PCT application serial number PCT/CA00/01078 filed Sep. 15, 2000 entitled APPARATUS AND PROCESS FOR CONDITIONING MAMMALIAN BLOOD (the entire contents of which are incorporated herein by reference). Alternatively, one treatment device may be equipped to perform a range of invasive and non-invasive treatments such as surgeries, treatments for diseases such as cancer, as well as exploratory or diagnostic investigations such as X-rays, CAT Scans, MRI's and the like.

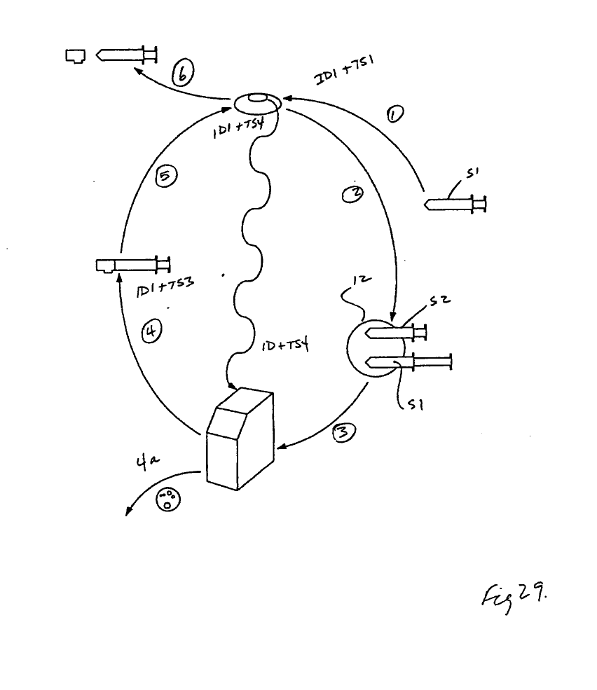

As will be described, the system provides a verification protocol which involves number of verification checks to be sure that the proper treated blood sample is delivered to the proper originating patient. To that end, and as shown in FIG. 26, the system has identification means 350 for identifying an originating patient “P”, for the untreated blood sample in syringe S1, verification means 352 for verifying a match between the originating patient “P”, and the treated blood sample in syringe S2, and release signal generating means 354 for generating a release signal in response to a positive verification by the verification means. The release signal is conveyed to the releasable lock means 220 to deliver the predetermined current to the ring 314, thereby to render syringe S2 operable to deliver the treated blood sample to the originating patient.

As will be described, the identification means 350 and the release signal generating means 354 are located on the wrist band 16. The releasable lock means has a signal receiving means 358 for receiving the release signal. At least some of the functions of the verification means 352 are also included in the wrist band 16 as will be described.

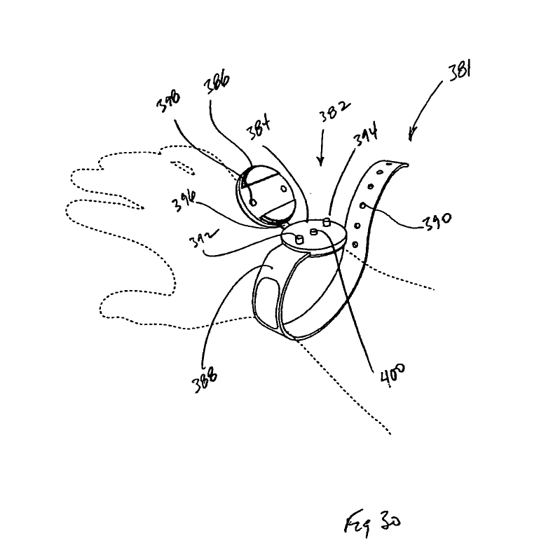

Referring to FIG. 27, the verification means 352 includes comparison means 360 for comparing patient identity data with treated blood sample identity data, both stored in memory means 362, and signal receiving means 364 to receive one or more signals associated with the originating patient identity data and/or the blood sample identity data (either untreated, treated or both). In this case, the one or more signals contain the originating patient identity data and/or the blood sample identity data. However, as an alternative, the one or more signals may contain data which is associated with or related to the patient or blood sample identity data. For example, the data in the signals may include one or more codes which allow the patient identity data or the blood sample identify data to be obtained from a data structure in the memory means 362 or some other location, for example in the form of a look up table, for instance.

The memory means 362 may include time value data to determine at least one time value related to a predetermined event including and/or between an untreated blood sample collection event and a treated blood sample delivery event. The time value may also include at least one elapsed time value between two predetermined events including or between the untreated blood sample collection event and the treated blood sample delivery event. In this case, the verification means may be operable to prevent release of the locked third fluid coupling when the elapsed time value has exceeded a predetermined elapsed time maximum value.

Before treatment of the untreated blood sample, the verification means 352 is also operable to prevent treatment of the blood sample when the elapsed time value has exceeded a predetermined elapsed time maximum value. Similarly, following treatment, the verification means 352 is operable to verify a match between the untreated blood sample in the first syringe and the originating patient.

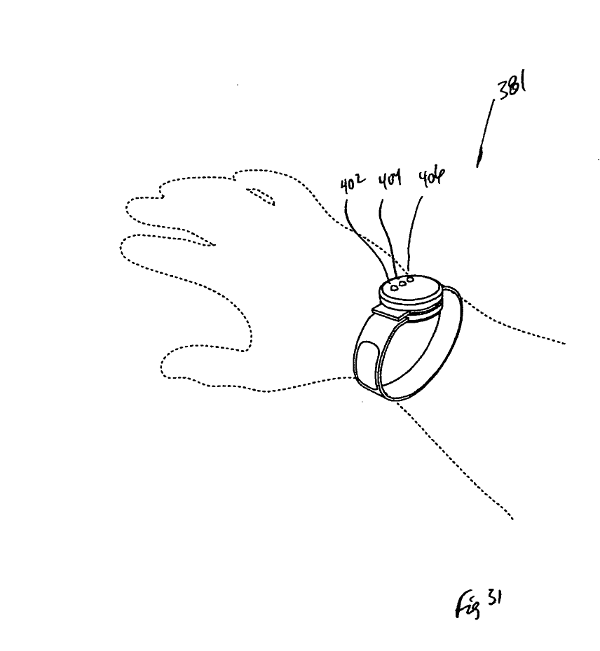

The verification protocol may be implemented in a number of forms, although the most preferred at present is by the use of one or more radio frequency signal transmitters and receives, in chip or chipless form and popularly referred to as RFID chips or tags. In this case, as shown in FIG. 28, the wrist band 16 is provided with an “active” WB RFID chip 370 while the syringes S1 and S2 are both provided with “passive” 51 RFID chip 372 (see also FIG. 3) and S2 RFID chip 374 (see also FIG. 16) respectively. The term “active” refers to the ability of the WB RFID chip 370 to send query signals to the S1 RFID chip 372 and the S2 RFID chip 374, both of which are operable in response to the query signal, either to emit a signal or to receive and record data. The WB RFID chip 370 is active in that it issues query signals to the S1 RFID chip 372 to write untreated blood sample identity/verification data thereon.

There are a number of RFID chips currently available in both “active” and “passive” chip and chipless form, including those of the chip form under the trade names (TEXAS INSTRUMENTS), ISO 15693 RE Tag inlay; MELEXIS, MLX90127 transponder; PHILIPS, HT1DC2OS3O transponder; and Microchip, MCRF45S chip, and including those of the chipless form under the trade names SENSORMATIC, Ultra.Strip® III; and CHECKPOINT's EAS tags and labels. Currently, several commercial chip tags are not able to stand the gamma radiation used for medical component sterilization. Chipless tags are usually better than chip tags in terms of withstanding gamma radiation. However, the chip tags tend to be more attractive in view of their relatively higher data carrying capacity. It is contemplated that the sensitivity to gamma radiation may be addressed by a employing a relatively harder coating for chip tags, that is to package the chip tags with gamma hardening technology so that the tags will be able to stand gamma radiation. Alternatively, the advances in chipless tags may improve the amount of data they can carry as well as the ability to write the data on them. Other sterilization methods may also be appropriate, using alternative sterilizing atmospheres such as EtO (ethylene oxide).

The blood treatment unit 14 is also equipped with RE communication, by way of an active RFID chip 376 to receive a pre-treatment identity data from the S1 RFID chip 372 and to write post treatment data to the 52 RFID chip 374. Similarly, the blood treatment chamber 12 is equipped with an RFID chip 378 to provide an identification code for reasons to be described.

Referring to FIG. 28, the wrist band 16 contains a removable portion 380 containing the WB RFID chip 370 and audit data written onto it relating to the patient and/or the treated blood sample.

Alternatively, the wrist band may be provided with an activation tab. For example, the activation tab may be included on the wrist band which must be removed, severed or disabled in some manner in order to couple the wrist band on the patient.

Another alternative is shown in FIGS. 30 to 32, wherein a wrist band 381 includes a buckle assembly 382 having a base portion 384 and cover portion 386. The base portion 384 is integrally formed with a band 388 of resilient material which a number of perforations forming passages 390 to receive the buckle assembly 382. The base portion 384 has a pair of pins 392, 394 which are dimensioned to fit through the passages 390. The cover portion 386 is hinged to the base portion 384 by way of a living hinge shown at 396. The cover portion 386 also has a pair of cavities 398, each for receiving one of the pins 392. Located between the pins 392, 394 is an activation button 400 which is moveable from its extended position above the base to an activating position flush with the base when the strap is located thereon. When in the activating position, the activation button 400 is operable to power up the RFID chip 370 to begin broadcasting query signals intended for S1. The cover portion is also provided with a number, in this case three, LED indicator lights 402, 404, 406 which are operated in different combinations of one or more thereof. Two LED's 402, 404 may be green in colour, the LED 402 for syringe S1 and the LED 404 for syringe 52. Each LED may be operable to blink in one phase indicating that the verification protocol is either at the S1 or S2 processing steps. The third LED 406 may be provided for alarm situations. The wrist band 381 also has a removable portion 408 containing an RFID chip and, following treatment, the audit data therein.

The verification protocol involves a number of identification codes as follows. The first syringe S1 is assigned a first syringe identity code which is representative of the untreated blood sample therein, and the wrist band 16 is assigned a wrist band identity code which is representative of the originating patient. To simplify the data transfer, the first syringe and wrist band identity codes may include common data, though the data between them may be different or related as the case may be. The first syringe identity code may, if desired, include a first time value representative of the time of untreated sample collection from the originating patient (or a designated event either before or after the sample collection step) and/or verification thereof.

Thus the S1 RFID chip 372 functions as a first signal emitter for emitting a first signal carrying the originating patient identity data, while the WB RFID chip 370 on the wrist band 16 functions as a first signal receiver to receive the first signal. The second syringe 52 is assigned a second syringe identity code, which is representative of the treated blood sample therein. The second syringe identity code includes a second time value representative of the time of the treated sample delivery thereto from the blood sample treatment chamber 12 (or a designated event either before or after the treated sample delivery step) and/or verification thereof.

Thus, the S2 RFID chip 374 functions as a second signal emitter for emitting a second signal carrying the treated blood sample identity data and the WB RFID chip 370 functions as a second signal receiver means to receive the second signal, wherein the verification means is operable to compare the first signal data with data representative of the treated blood sample.

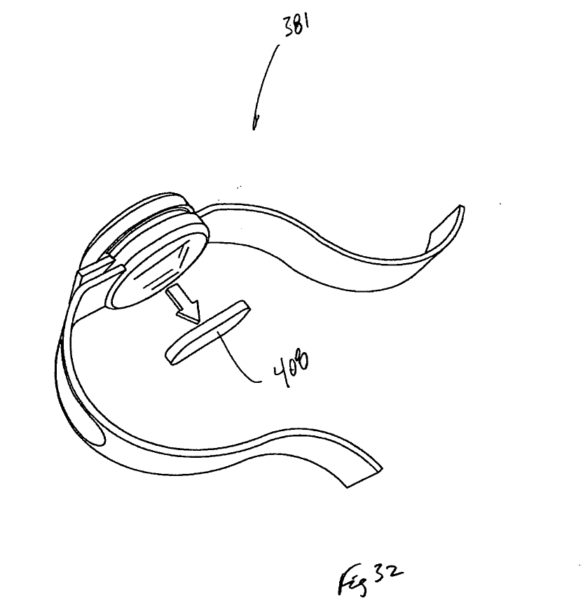

Referring to FIG. 29, the verification protocol will now be discussed together with a typical blood treatment procedure.

First, a package is assembled including, among other things, one wrist band 16, one SI syringe, one S2 syringe, one sample treatment chamber 12 and a number of prepared labels (shown at 410 in FIG. 1) with patient identification printed thereon. The WB RFID chip 370 is then activated for use. In this particular example, the S1 RFID chip 370 and the WB RFID chip 372 each contain common patient identity data coded as ID 1. The syringe S1 is prepared for a sample by first injecting a solution such as sodium citrate into the syringe, as shown in FIG. 7.

The syringe S1 is then used to draw a sample of blood. Once filled, the S1 syringe is brought to within RE range of the wristband, whose WB RFID chip 370 verifies that the data read from or emitted by the 51 RFID chip 370 corresponds to the patient identify data ID 1. Once a positive correlation has been made, the WB RFID chip 370 writes a “time data stamp” TS1 stamp on the S1 RFID chip 372, so that it now carries both Di+TS1. In this case, the TS1 data is the time count at that instant.

The WB RFID chip 370 functions by issuing regular query signals to syringe S1. S1 will eventually acknowledge the query signal and with a return signal containing S1 ID 1 data which is compared with the wrist band ID1 data. Then, once a positive correlation has been made, the WB RFID chip 370 issues a write signal which includes the ID1 data as its “header” and the time stamp TS1 in its “payload” (the “header” and “payload” being well known components of TS1 signals of this type). The TS1 data will change with increasing delay, so that the TS1 signal will be different depending on when the filled S1 syringe returns to the wristband following blood sample collection. The wristband then measures the elapsed time from the start of the procedure (that is TS0, which, in this case, is the instant that the WB is activated) and the point at which the S1 RFID chip acknowledges the query signal. In this case, the WB RFID chip 370 may, if desired, halt the process if the elapsed time between TS0 and TS1 exceeds a predetermined maximum time period.

For example, the code now in the S1 RFID chip 372 may be represented as: S1 ID1 12/31/03 14:00 meaning that the sample in S1 is from patient ID1 and the sample collection was recorded at Dec. 31, 2003 at 1400 hours.

The time data TS1 may be in any time measure but is conveniently based on “Internet Time” or on a time standard such as Greenwich Mean time (GMT), or alternatively may be an elapsed time count.

After the S1 RFID chip 372 receives the TS1 data, the S1 syringe is installed on the chamber (with the S2 syringe S2 also positioned thereon) which is then delivered to the Blood Treatment Unit (or BTU) 14. Here, the S1 RFID chip 372 receives a query signal from the BTU RFID chip 376 and, in response thereto, emits the data ID1+TS1. The BTU 14 then calculates the time delay between TS1 and the arrival time of S1. In addition, the BTU issues a query signal to the RFID chip 378 on the blood treatment chamber 12 and, in response thereto, the RFID chip 378 issues a signal containing its identification code to the BTU. This identification code, in this case, includes an “enable” code indicating that the treatment chamber 12 has not been previously used for a blood treatment, thus reducing the risk of contamination the current untreated blood sample SI. Alternatively, the RFID chip 378 need not issue an enable code, but rather merely emit a signal containing identity data such as a stock number or the like.

Having calculated the time delay, the BTU then determines if the time delay has exceeded a predetermined maximum value, and if so the BTU 14 shuts down the procedure. Otherwise, the ID1 and TS1 data from the syringe S1 is recorded in the BTU and the procedure continues with the untreated blood sample in the S1 syringe being delivered to the treatment cavity 82, by way of an actuator in the BTU depressing the plunger on syringe S1i. The BTU 14 then disables the S1 RFID chip 374, by writing a disable code thereon. In addition, the RFID chip 378 on the blood treatment chamber 12 contains an identification code and receives a disable code from the BTU 14 when or after the blood sample is delivered to it, thereby preventing the treatment chamber 12 from being used again. Alternatively, the RFID chip 378 may be disabled in other ways without writing a disable code thereon. For example, the RFID chip 378 may be rendered inoperable using other techniques such as by issuing the RFID chip 378 a signal causing a fuse to be blown therein.

The BTU 14 then proceeds to treat the blood sample which is then delivered to S2. The BTU then writes the D1 data together with a new time stamp signifying the end of the blood sample treatment “TS3” to the 52 RFID chip 374. If desired, the BTU 14 may also include the 151 stamp, meaning that the data written to the S2 RFID chip 374 would include ID1+TS1+TS2+TS3. In this case, 152 includes the treatment start time and TS3 includes the treatment end time. Alternatively, or in addition, 1S2 or TS3 may include a treatment duration time, or some other code indicating that all previous verification steps have been successfully carried out.

For example, the BTU may record the following data:
S1 D1 12/31/03 14:00
PATIENT ID
TREATMENT START 12/3 1/03 14:02
TREATMENT END 12/3 1/03 14:20
S2 ID1 12/31/03 14:20.

In this case, the PATIENT ID code may include other patient coordinate information that is manually or automatically entered into the BTU or alternatively data which is transferred to the BTU from a central data storage centre, a server computer a memory bank or the like.

In this case, the BT'LT may then record in the S2 RFID chip 374:
S2 ID1 12/31/03 14:20

The syringe S2 is then transported back to the originating patient wearing the wristband and the WB RFID chip 370 continually polls the 52 RFID chip 374 until the latter is within range and then emits D1 data, subsequently read by the WB RFID chip 370, together with the 153 data. The wristband then calculates the time delay between 153 data and the time of arrival of 52 back to the wristband. If the expected time day is exceeded, the wristband does not permit the S2 syringe to function.

The wristband records ID1, and a time stamp “TS4” which signifies the verification and ID1 confirmation. In addition, the wrist band may also record the PATIENT ID data as well as the ID1+TS1+TS2+TS3, if desired. At this stage, the WB RED chip 370 issues a release signal to the S2 RFID chip 374 which, on receipt thereof, issues a predetermined current on the ring 314 to release the locking pin 304, thereby rendering S2 operable for injection.

For example, the WB RFID chip 370 may therefore record:
S1 ID1 12/31/03 14:00
S2 ID1 12/31/03 14:20
SAMPLE MATCH 12/3 1/03 14:30
S2 UNLOCK 12/31/03 14:30.

The verification protocol is then completed when the TS4 is recorded in the WB RFID chip 370 after it performs a sample match between the ID1 data on the S2 RFID chip 374 and the WB RFID chip 370. Then, the WB RFID chip 370 adds the TS4 data to the ID1 data (and if desired, the PATIENT ID data and any one of the TS0, TS1, TS2, and TS3 data). The removable portion of the wristband is then separated therefrom and matched with the originating patient's record and the patient record is returned to the BTU for a data exchange between the WB RFID chip 370 and the BTU 14.

Alternatively, an RF reading audit record capture station may be provided which is be local to the patient or to a patient record area n the medical facility, thereby eliminating the need for the patient record to be returned to the BTU. In this case, the audit record capture station may be capable of downloading the patient record to complete the audit trail. The RF reading audit record capture station may be part of the internal network of the medical facility, either through a wired or wireless data port, or may be part of a network localized to one or BTU systems in the medical facility. It may collect data and allow for later batch recording to a medium such as a compact disk or other memory or storage device. It may be a attached to or integrally formed with a notebook computer, personal data assistant, cell phone or the like. It may also be embodied in software configured to run on a computer, together with an RF reading attachment thereon.

On the other hand, the amount of data may be reduced, simply by providing the TS3 data to the BTU which matches it with the ID1 data, by relying on the fact that the TS4 indicates that ID1 data must match, because TS4 exists only because a match was made between the original WB ID data and the S2 data.

The audit trail is then completed by ID1 and TS4 being delivered to BTU or other system.

The time stamp may also include an “event” code, which may comprise five major events:
1) WB start time
2) 51 acknowledge with WB
3) Start of Treatment
4) End of Treatment
5) Match between the Treated Sample and the Originating Patient.

The time stamp may also include any one or more of a number of Error events
1) No match
2) S1 does not match with WB at before/after collection
3) S2 does not match with WB on return after Treatment.
4) Time Delay—exceed time to collect of blood
5) Time Delay—exceed time to deliver sample to BTU

6) Time Delay—exceed time to return to patient.

The 153 time stamp may also include a “match” code as follows:
01 Match
02 No match

The identification means verification means and/or the release signal generating means may be located in several possible locations. For example, verification means and/or the release signal generating means may be located on the second syringe S2, for example within the lock housing 306. In this case, the S2 RFID chip 374 may be active to issue query signals to the wrist band 16 to receive a signal therefrom containing a WB ID signal, and thereafter conduct a comparison between the WB ID data and the ID1 data.

Alternatively, the verification means, the identification means and/or the release signal generating means may be located on the on the blood sample transfer portion or the blood treatment unit.

The wrist band 16 may be replaced by some other article to be warn, carried, attached or ingested by the patient, such as a pinned or self adhesive label and the like.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

1. A system for the collection, treatment and delivery of an autologous blood sample, comprising:
a first syringe having a first body portion, a first sample transfer portion having a first syringe inlet for drawing an untreated blood sample from a patient and a first syringe outlet for dispensing the untreated blood sample;
a blood sample treatment chamber having a chamber inlet, the first syringe outlet being operable to establish a dedicated first fluid coupling with the chamber inlet to dispense the untreated blood sample to the blood sample treatment chamber;
the blood sample treatment chamber having a chamber outlet for dispensing a treated blood sample following treatment;
a second syringe having a second body portion and a second sample transfer portion, the second transfer portion having a passage with a first access location which is operable to form a dedicated second fluid coupling with the chamber outlet to receive a treated blood sample from said blood sample treatment chamber;
the second body portion having a second syringe outlet;
locking means associated with said second syringe to prevent dispensing of treated blood from said second syringe in the absence of a release signal;
identification means for identifying an originating patient for the untreated blood sample;
verification means for verifying a match between the originating patient and the treated blood sample;
and release signal generating means for generating said release signal in response to a positive verification by the verification means.

2. A system as defined in claim 1 further including:
said passage in said second transfer portion having a second access location for fluid communication with said second syringe outlet;
said locking means forming a locked third fluid coupling between said second access location;
said locking means being operable in response to said release signal to release said third fluid coupling; and
said second syringe outlet being operable when released from said third fluid coupling to form a fourth fluid coupling with a blood sample delivery unit.

3. A system as defined in claim 1 wherein the identification means and/or the release signal generating means is located on the second syringe body.

4. A system as defined in claim claim 1 wherein the identification means and/or the release signal generating means is located on the on the second sample transfer portion, the locking means further comprising signal receiving means for receiving the release signal.

5. A system as defined in claim 1 wherein the identification means and/or the release signal generating means is located on an external article, the locking means further comprising signal receiving means for receiving the release signal.

6. A system as defined in claim 5 wherein the external article is carried by or worn by the originating patient.

7. A system as defined in claim 6 wherein the external article includes a wrist band.

8. A system as defined in claim 6 wherein the external article contains a removable portion containing audit data relating to the patient and/or the treated blood sample.

9. A system as defined in claim 1 wherein the verification means includes comparison means for comparing originating patient identity data with the treated blood sample identity data, signal receiving means to receive one or more signals associated with the originating patient identity data and/or the blood sample identity data, and memory means for storing the patient identity data and the treated blood sample identity data.

10. A system as defined in claim 9 wherein the one or more signals contain the originating patient identity data and/or the blood sample identity data.

11. A system as defined in claim 9, wherein the memory means stores time value data to determine at least one time value related to a predetermined event including or between an untreated blood sample collection event and a treated blood sample delivery event.

12. A system as defined in claim 11 wherein the time value includes at least one elapsed time value between two predetermined events including or between the untreated blood sample collection event and the treated blood sample delivery event.

13. A system as defined in claim 12 wherein the verification means is operable to prevent release of the locked third fluid coupling when the elapsed time value has exceeded a predetermined elapsed time maximum value.

14. A system as defined in claim 12, further comprising a blood treatment unit to treat the blood sample in the blood sample treatment chamber, the verification means being operable to prevent treatment of the blood sample when the elapsed time value has exceeded a predetermined elapsed time maximum value.

15. A system as defined in claim 9 wherein the verification means is operable to verify a match between the untreated blood sample in the first syringe and the originating patient.

16. A system as defined in claim 15 wherein the first syringe is assigned a first syringe identity code which is representative of the untreated blood sample therein, and the originating patient is assigned an originating patient identity code which is representative of the originating patient, wherein the first syringe and originating patient identity codes include related or common data.

17. A system as defined in claim 16 wherein first syringe identity code includes a first time value representative of the time of untreated sample collection from the originating patient and/or verification thereof.

18. A system as defined in claim 16 wherein the second syringe is assigned a second syringe identity code, which is representative of the treated blood sample therein.

19. A system as defined in claim 18 wherein the second syringe identity code includes a second time value representative of the time of the treated sample delivery thereto from the blood sample treatment chamber and/or verification thereof.

20. A system as defined in claim 1 wherein the identification means includes a first signal emitting means for emitting a first signal carrying untreated blood sample identity data and a first signal receiving means to receive the first signal.

21. A system as defined in claim 20 wherein the first signal emitting means is located on the first syringe.

22. A system as defined in claim 1 wherein the identification means includes a second signal emitting means for emitting a second signal carrying the treated blood sample identity data and a second signal receiving means to receive the second signal.

23. A device as defined in claim 22 wherein the second signal emitting means is located on the second syringe.

24. A system as defined in claim 1 wherein the first body portion of the first syringe includes an untreated blood sample receiving chamber and the first sample transfer portion includes a passage joining the untreated blood sample receiving chamber with the first syringe inlet and the first syringe outlet, and first syringe inlet valve means for controlling the flow of blood through the first syringe inlet.

25. A system as defined in claim 24, the first syringe inlet valve means including a septum, further comprising a complementary penetrating member located on or intermediate to the external blood collection article and in an engaged position therewith.

26. A system as defined in claim 1 wherein the second blood sample transfer portion includes a filtered vent outlet in the passage for expelling one or more gas constituents in the treated blood sample.

27. A system as defined in claim 1 wherein one or both of the dedicated first and second couplings are operable releasably to lock the first sample transfer portion of the first syringe and the second sample transfer portion of the second syringe with the blood sample treatment chamber in respective open fluid transfer conditions.

28. A system as defined in claim 27 wherein one or both of the dedicated first and second couplings are operable to establish the locked open fluid transfer condition upon relative rotational displacement between the blood sample treatment chamber and the corresponding first and second sample transfer portions.

29. A system as defined in claim 1 wherein the second syringe outlet includes second syringe outlet valve means for controlling the flow of the blood sample there through.

30. A system as defined in claim 29 wherein the second syringe outlet valve means includes a valve element potion and a valve seat portion, and actuating means for actuating the valve element portion relative to the valve seat portion, the actuating means being operable to displace the valve element from the valve seat portion when the second body portion is engaged with the second sample transfer portion.

31. A system as defined in claim 30 wherein the second sample transfer portion includes a housing to receive the second syringe outlet therein, the housing having a female member in fluid communication with the second access location, the second syringe outlet including a male portion to engage the female portion, the actuating means including an actuating portion adjacent the male portion to be displaced by the female portion on engagement of the female portion with the male portion.

32. A system as defined in claim 31, further comprising an outer sheath portion spaced from the male portion to form an annular female portion-receiving passage therein, the actuating portion including at least one first actuating element positioned in the annular passage.

33. A system as defined in claim 29 wherein the second syringe outlet valve means includes a valve element portion and a valve seat portion, and actuating means for actuating the valve element portion relative to the valve seat portion, the actuating means being operable to engage the valve element with the valve seat portion when the second body portion is separated from the second sample transfer portion, and a second syringe outlet end portion extending outwardly from the second body portion.

34. A system as defined in claim 33 wherein the actuating portion includes at least one second valve actuating element which extends laterally outwardly beyond the second syringe outlet end portion.

35. A system as defined in claim 34 wherein the second outlet end portion has a bevelled distal end and the second valve actuating element has a distal end region which is configured to engage the bevelled distal end of the second outlet end portion.

36. A system as defined in claim 35 wherein the distal end region of the second valve actuating element is angled to nest with the bevelled distal end of the second outlet end portion when the valve element portion is engaged with the valve seat portion.

37. A system as defined in claim 36 wherein the second valve actuating element is arranged to travel along an outside surface of the second outlet end portion as the valve portion is displaced relative to the valve seat portion.

38. A system as defined in claim 37, further comprising a collar member located within the housing, the collar member including a chamber to receive the second outlet end portion to form the third fluid coupling.

39. A system as defined in claim 38 wherein the second valve actuating element includes an abutment flange extending outwardly therefrom, the abutment flange being operable to abut a designated location in the chamber when the second syringe outlet is removed from the chamber.

40. A system as defined as claim 38 wherein the releasable lock means includes a barrier member positioned adjacent the second access location and moveable between a locked position in which the barrier member engages the second outlet end portion, and a release position in which the barrier member is retracted from the second outlet end portion.

41. A system as defined in claim 40 wherein the barrier member is biased to the release position.

42. A system as defined in claim 41, further comprising a brace means for bracing the barrier member in the locked position, wherein the brace means is releasable in the presence of a predetermined current.

43. A material dispensing device, comprising a material container portion and a material transfer portion, the material transfer portion including a passage for the transfer of materials to and from the material container portion, the passage having a first access location in fluid communication with the material container portion and a second access location, and second access location control means for controlling the flow of material through the second access location, the second access location control means including a penetrable septum which is operable in an unpenetrated condition in which the passage is closed and a penetrated condition in which the passage is open, and a third access location, the third access location including a means for forming a dedicated fluid coupling with a medical materials dispenser.

44. A device as defined in claim 43 wherein the material container portion is integrally formed with the material transfer portion.

45. A device as defined in claim 44 wherein the material is a medical fluid.

46. A device as defined in claim 43 wherein the septum includes a block of resilient material.

47. A device as defined in claim 46, the block having a diameter and a depth, wherein the depth approximates the diameter.

48. A device as defined in claim 47, further comprising a septum housing portion containing the septum, the device further comprising a penetrating member for penetrating the septum, the penetrating member being associated with a flange which is engageable with the septum housing portion.

49. A device as defined in claim 48 wherein the flange is complementary with an outer surface on the housing portion.

50. A device as defined in claim 48 wherein the septum housing portion includes an outer web spaced therefrom to form a peripheral cavity to receive the flange on the material transfer portion.

51. A device as defined in claim 43 wherein, in the engaged position, fluid communication is enabled through the penetrating member.

52. A device as defined in claim 51 wherein the penetrating member is a hollow or grooved spike member.

53. A device as defined in claim 52 wherein the septum is located adjacent an end flange, the end flange having an opening with a predetermined cross section to match the cross section of the spike member.

54. A device as defined in claim 53, further comprising an inner septum passage adjacent the end flange, at least one lock member movable between an operable position to obstruct the inner septum passage and an inoperable position, the lock member further including displacement means for displacing the locking member to the inoperable position by the presence of the spike member of a minimum lateral dimension in the inner septum passage.

55. A device as defined in claim 54, further comprising a pair of lock members, each of which includes an outer lock flange and wherein, in the operable position, the lock flanges overlap one another.

56. A device as defined in claim 55 wherein the displacement means includes shank portions located on each lock member adjacent the outer lock flange, the shank portions arranged to lie adjacent one another in the inner septum passage in the operable position, the shank portions being movable to the inoperable position when the spike member of minimum lateral dimension is introduced between the shank portions.

57. A device as defined in claim 48 further including a material delivery portion on which the penetrating member is provided.

58. A device as defined in claim 57 wherein the material delivery portion further comprises a base supporting the penetrating member and the flange on one side thereof.

59. A device as defined in claim 58 wherein the penetrating member is a hollow or grooved spike member and the base further comprises a conduit positioned on a side opposite the spike and in fluid communication therewith.

60. A device as defined in claim 59 wherein the material delivery portion includes a needle and/or needle catheter in fluid communication with the conduit.

61. A device as defined in claim 43 wherein the material container portion includes one or more syringes, IV bottles, powder and/or atomized fluids and/or gas inhalant dispensers, implant delivery dispensers, ventilators, syringe pumps, intubation tubes, gastrointestinal feeding tubes, or a plurality and/or a combination thereof.

62. A material dispensing device, comprising a chamber and an outlet, valve means for controlling the outlet, the valve means including a penetrable septum which is operable in an unpenetrated condition in which the chamber is closed and a penetrated condition in which the chamber is open, the septum including a block of resilient material having a diameter and a depth, wherein the depth approximates the diameter, an end flange, the end flange having an opening with a predetermined cross section, and a penetrating member for penetrating the septum to open the chamber, the penetrating member having a matching cross section which matches the cross section of the opening in close fitting relationship therewith, wherein the opening prevents access to the septum by penetrating members without the matching cross section.

63. A material dispensing device, comprising a chamber and an outlet, valve means for controlling the outlet, the valve means including a penetrable septum which is operable in an unpenetrated condition in which the chamber is closed and a penetrated condition in which the chamber is open, the septum including a block of resilient material having a diameter and a depth, wherein the depth approximates the diameter, the septum having an inner septum passage, at least one lock member movable between an operable position to obstruct the inner septum passage and an inoperable position, the lock member further including displacement means for displacing the locking member to the inoperable position by the presence of a penetrating member of a minimum lateral dimension in the inner septum passage.

64. A device for controlling a medical materials dispenser, comprising a control portion, the control portion having a housing with a passage therein, the passage forming a first fluid coupling with a delivery outlet portion on the medical materials dispenser, and a second fluid coupling with a medical materials receptacle; and releasable locking means for locking the first fluid coupling, the lock means being operable between a locked condition and unlocked condition in response to an actuation signal generated by an external device.

65. A device as defined in claim 64 wherein the medical materials dispenser includes one or more syringes, JV bottles, powder and/or atomized fluids and/or gas inhalant dispensers, implant delivery dispensers, ventilators, syringe pumps, intubation tubes, gastrointestinal feeding tubes, or a plurality and/or a combination thereof.

66. A device as defined in claim 64 wherein the passage includes a gas discharge vent to permit the gas from the medical materials dispenser to be discharged there through.

67. A system as defined as claim 64 wherein the releasable lock means includes a barrier member moveable between a locked position in which the barrier member engages the delivery outlet portion and a release position in which the barrier member is retracted from the delivery outlet portion.

68. A system as defined in claim 67 wherein the barrier member is biased to the release position.

69. A system as defined in claim 68, further comprising a brace means for bracing the barrier member in the locked position, wherein the brace means is releasable in the presence of a predetermined current.

70. A syringe device comprising a syringe body, the syringe body having a first body portion with a cavity formed therein, a plunger in sealed engagement with the cavity to form a fluid receiving chamber, the syringe body having a second body portion, the second body portion having a passage formed therein, the passage having a first access location in fluid communication with the chamber and a second end terminating at a second access location, the passage having a third access location, wherein at least one of the second and third access locations includes a penetrable septum which is operable in an unpenetrated condition in which the passage is closed and in a penetrated condition in which the passage is open.

71. A device as defined in claim 70 wherein the first syringe body is integrally formed with the second syringe body.

72. A device as defined in claim '70 wherein the septum includes a block of resilient material, having a diameter and a depth, wherein the depth approximates the diameter.

73. A device as defined in claim 72 wherein the second syringe body portion includes a septum housing portion containing the septum, the device further comprising a penetrating member for penetrating the septum, the penetrating member being associated with a flange which is engageable with the septum housing portion.

74. A device as defined in claim 73 wherein the flange is complementary with an outer surface on the housing portion.

75. A device as defined in claim 74 wherein, in the engaged position, fluid communication is provided through the penetrating member.

76. A method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
collecting the sample in a second collection device;
associating the second collection device with a third signal carrying data representative of the processed sample;
comparing the data in the first and third signals to link the processed sample with the patient; and thereafter;
permitting the processed sample to be released after a positive association of the data.

77. A method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
collecting the processed sample in a second collection device, wherein the second collection device is locked against release of the process sample in the absence of an unlocking signal;
associating the second collection device with a third signal carrying data representative of the processed sample;
comparing the data in the first and third signals to link the processed sample with the patient; and thereafter;
unlocking the second collection device to permit release of the processed sample when an unlocking signal is sent after a positive association has been made between the processed sample and the patient.

78. A method of monitoring a material sample from a patient, comprising the steps of,
collecting the sample from the patient with a first collection device;
associating the patient with a first signal carrying data representative of the sample;
associating the first collection device with a second signal carrying data representative of the sample;
delivering the sample to a sample treatment chamber;
processing the sample to form a processed sample;
associating the processed sample with a third signal carrying data representative of the processed sample;
comparing the data in the first and third signals to link the sample as processed with the patient and thereafter;
assembling a patient record including the data in one or more of the first, second and third signals.
